SEQUENCE LISTING (28 sequences)

SEQ ID NO: 1 - DNA, 41 bp, Artificial Sequence
  Description of Artificial Sequence Synthetic oligonucleotide
  Backbone of puromycin linker
aaaaaaaaaaaaaaaaaaaasttccagccgccccccgvcct

SEQ ID NO: 2 - DNA, 274 bp, Artificial Sequence
  Description of Artificial Sequence Synthetic polynucleotide
  Rundom library sequence
  modified_base (124)..(125): a, c, g, or t
  modified_base (127)..(128): a, c, g, or t
  modified_base (130)..(131): a, c, g, or t
  modified_base (133)..(134): a, c, g, or t
  modified_base (136)..(137): a, c, g, or t
  modified_base (139)..(140): a, c, g, or t
  modified_base (142)..(143): a, c, g, or t
  modified_base (145)..(146): a, c, g, or t
gatcccgcgaaattaatacgactcactataggggaagtatttttacaacaattaccaacaacaacaacaaacaacaacaacattacattttacattctacaactacaagccaccatgggcagcnnknnknnknnknnknnknnknnkggaggtggaattaaaaacatgtgcaatttgaacccacttttaaaaaagtggctaaatgatgcaaaggggggaggcagccatcatcatcatcatcacggcggaagcaggacggggggcggcgtggaaa

SEQ ID NO: 3 - DNA, 60 bp, Artificial Sequence
  Description of Artificial Sequence Synthetic oligonucleotide
  Sequence of primer, T7omega-new
  primer_bind (1)..(60)
gatcccgcgaaattaatacgactcactataggggaagtatttttacaacaattaccaaca

SEQ ID NO: 4 - DNA, 39 bp, Artificial Sequence
  Description of Artificial Sequence Synthetic oligonucleotide
  Sequence of NewYtag
  primer_bind (1)..(39)
tttccacgccgccccccgtcctgcttccgccgtgatgat

SEQ ID NO: 5 - DNA, 33 bp, Artificial Sequence
  Description of Artificial Sequence Synthetic oligonucleotide
  Sequence of Newleft
  primer_bind (1)..(33)
gatcccgcgaaattaatacgactcactataggg

SEQ ID NO: 6 - DNA, 367 bp, Staphylococcus aureus
gatcccgcgaaattaatacgactcactataggggaagtatttttacaacaattaccaacaacaacaacaaacaacaacaacattacattttacattctacaactacaagccaccatggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacgggggaggcagccatcatcatcatcatcacggcggaagcaggacggggggcggcgtggaaa

SEQ ID NO: 7 - DNA, 541 bp, Artificial Sequence
  Description of Artificial Sequence Synthetic polynucleotide
  DNA construct for VHH library
  modified_base (199)..(199): a, c, t or g
  modified_base (200)..(200): t or c
  modified_base (201)..(202): a, c, t or g
  modified_base (203)..(203): t or c
  modified_base (204)..(206): a, c, t or g
  modified_base (207)..(207): t or c
  modified_base (208)..(209): a, c, t or g
  modified_base (210)..(210): t, c or g
  modified_base (211)..(212): a, c, t or g
  modified_base (213)..(213): t, c or g
  modified_base (214)..(215): a, c, t or g
  modified_base (216)..(216): t, c or g
  modified_base (217)..(218): a, c, t or g
  modified_base (219)..(219): t or c
  modified_base (220)..(220): a, c, t or g
  modified_base (221)..(221): t or c
  modified_base (222)..(223): a, c, t or g
  modified_base (224)..(224): t or c
  modified_base (225)..(225): a, c, t or g
  modified_base (265)..(265): a, c, t or g
  modified_base (266)..(266): t or c
  modified_base (267)..(268): a, c, t or g
  modified_base (269)..(269): t or c
  modified_base (270)..(272): a, c, t or g
  modified_base (273)..(273): t or c
  modified_base (274)..(275): a, c, t or g
  modified_base (276)..(276): t, c or g
  modified_base (277)..(278): a, c, t or g
  modified_base (279)..(279): t, c or g
  modified_base (280)..(281): a, c, t or g
  modified_base (282)..(282): t, c or g
  modified_base (283)..(284): a, c, t or g
  modified_base (285)..(285): t, c or g
  modified_base (286)..(287): a, c, t or g
  modified_base (288)..(288): t or c
  modified_base (412)..(413): a, c, t or g
  modified_base (414)..(414): t or c
  modified_base (415)..(416): a, c, t or g
  modified_base (417)..(417): t or c
  modified_base (418)..(419): a, c, t or g
  modified_base (420)..(420): t, c or g
  modified_base (421)..(422): a, c, t or g
  modified_base (423)..(423): t, c or g
  modified_base (424)..(425): a, c, t or g
  modified_base (426)..(426): t, c or g
  modified_base (427)..(428): a, c, t or g
  modified_base (429)..(429): t, c or g
  modified_base (430)..(431): a, c, t or g
  modified_base (432)..(432): t, c or g
  modified_base (433)..(434): a, c, t or g
  modified_base (435)..(435): t, c or g
  modified_base (436)..(437): a, c, t or g
  modified_base (438)..(438): t, c or g
  modified_base (439)..(440): a, c, t or g
  modified_base (441)..(441): t, c or g
  modified_base (442)..(443): a, c, t or g
  modified_base (444)..(444): t, c or g
  modified_base (445)..(446): a, c, t or g
  modified_base (447)..(447): t, c or g
  modified_base (448)..(449): a, c, t or g
  modified_base (450)..(450): t or c
  See specification as filed for detailed description of substitutions and preferred embodiments
gatcccgcgaaattaatacgactcactataggggaagtatttttacaacaattaccaacaacaacaacaaacaacaacaacattacattttacattctacaactacaagccaccatgggcgaggtgcagctggtggagagcggaggaggatccgtgcaggctggaggaagcctgcgcctgagctgcgctgctagcggannnnnnnnnnnnnnnnnnnnnnnnnnntggttccgccaggctcctggaaaggagcgcgagggagtgnnnnnnnnnnnnnnnnnnnnnnnnacctactacgctgacagcgtgaagggacgcttcaccatcagccaggacaacgccaagaacaccgtgtacctgcagatgaacagcctgaagcctgaggacaccgctatctactactgcgctgctnnnnnnnnnnnnnnnnnnnnnnnnnnnnnnnnnnnnnnntactggggacagggaacccaggtgaccgtgggaggaggcagccatcatcatcatcatcacggcggaagcaggacggggggcggcggggaaa

SEQ ID NO: 8 - DNA, 32 bp, Artificial Sequence
  Description of Artificial Sequence Synthetic oligonucleotide
  Omega RT-L new
  primer_bind (1)..(32)
ggggaagtatttttacaacaattaccaacaac

SEQ ID NO: 9 - DNA, 22 bp, Artificial Sequence
  Description of Artificial Sequence Synthetic oligonucleotide
  NewYtag
  primer_bind (1)..(22)
tttccccgccgccccccgtcct

SEQ ID NO: 10 - DNA, 367 bp, Artificial Sequence
  Description of Artificial Sequence Synthetic polynucleotide
gatcccgcgaaattaatacgactcactataggggaagtatttttacaacaattaccaacaacaacaacaaacaacaacaacattacattttacattctacaactacaagccaccatggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacgggggaggcagccatcatcatcatcatcacggcggaagcaggacggggggcggcggggaaa

SEQ ID NO: 11 - DNA, 275 bp, Artificial Sequence
  Description of Artificial Sequence Synthetic polynucleotide
  DNA sequence coding flag sequence, wherein the sequence “NNKNNKNNKNNKNNKNNKNNKNNK” is replaced to “GATTATAAGGACGATGACGATAAG”.
gatcccgcgaaattaatacgactcactataggggaagtatttttacaacaattaccaacaacaacaacaaacaacaacaacattacattttacattctacaactacaagccaccatgggcagcgattataaggacgatgacgataaggggaggtggaattaaaaacatgtgcaatttgaacccacttttaaaaaagtggctaaatgatgcaaaggggggaggcagccatcatcatcatcatcacggcggaagcaggacggggggcggcgtggaaa

SEQ ID NO: 12 - DNA, 26 bp, Artificial Sequence
  Description of Artificial Sequence Synthetic oligonucleotide
  Description of Combined DNA/RNA Molecule Synthetic oligonucleotide
  Sequence of the conventional linker 1
  modified_base (5)..(5): biotin-dT
  modified_base (7)..(7): riboG
  modified_base (23)..(23): Amino-modifier C6 dT
ccgctcgccccgccgccccccgtcct

SEQ ID NO: 13 - DNA, 56 bp, Artificial Sequence
  Description of Artificial Sequence Synthetic oligonucleotide
  Sequence of a backbone of the conventional linker
  modified_base (53)..(53): a, c, g, or t
cccggtgcagctgtttcatctcggaaacagctgcaccccccgccgccccccgncct

SEQ ID NO: 14 - DNA, 28 bp, Artificial Sequence
  Description of Artificial Sequence Synthetic oligonucleotide
  I-Hybri segement of a linker of prior art.
  modified_base (3)..(3): Inosine, a, c, g, or t
  modified_base (8)..(8): a, c, g, or t
  modified_base (25)..(25): a, c, g, or t
ccncctcnaccccgccgccccccgncct

SEQ ID NO: 15 - DNA, 26 bp, Artificial Sequence
  Description of Artificial Sequence Synthetic oligonucleotide
  rG-Hybri segment of a linker
  modified_base (3)..(3): a, c, g, or t
  modified_base (5)..(5): a, c, g, or t
  modified_base (7)..(7): a, c, g, or t
  modified_base (23)..(23): a, c, g, or t
ccncncnccccgccgccccccgncct

SEQ ID NO: 16 - DNA, 42 bp, Artificial Sequence
  Description of Artificial Sequence Synthetic oligonucleotide
  Psoralen - amino segment for a linker
  modified_base (1)..(1): Psolaren C6 Phosphoramidite
  modified_base (39)..(39): Amino-modifier C6 dT
tacgacgatctcgaacgaaccacccccgccgccccccgtcct

SEQ ID NO: 17 - DNA, 28 bp, Artificial Sequence
  Description of Artificial Sequence Synthetic oligonucleotide
  Azid segment for a linker of prior art
cccgtggttcgttcgagatcgtcgtaaa

SEQ ID NO: 18 - protein, 12 aa, Artificial Sequence
  Description of Artificial Sequence Synthetic peptide
  Alkyne - peptide- biotin segment of a linker of a prior art
  MOD_RES (1)..(1): Gly(Propargyl)
  MOD_RES (12)..(12): Lys(Biotin)
GEDHVAHALAQK

SEQ ID NO: 19 - protein, 8 aa, Artificial Sequence
  Description of Artificial Sequence Synthetic peptide
  MOD_RES (1)..(8): Any amino acid
XXXXXXXX

SEQ ID NO: 20 - protein, 8 aa, Artificial Sequence
  Description of Artificial Sequence Synthetic peptide
  Converged 8 amino acid library at 3rd round.
DYKDDDDK

SEQ ID NO: 21 - RNA, 27 nt, Artificial Sequence
  Description of Artificial Sequence Synthetic oligonucleotide
  mRNA in Fig. 3B
gaagcaggacggggggcggcguggaaa

SEQ ID NO: 22 - DNA, 24 bp, Artificial Sequence
  Description of Artificial Sequence Synthetic oligonucleotide
  Nucleotide sequence of Sequence 19
  modified_base (1)..(24): a, c, g, or t
nnnnnnnnnnnnnnnnnnnnnnnn

SEQ ID NO: 23 - DNA, 22 bp, Artificial Sequence
  Description of Artificial Sequence Synthetic oligonucleotide
tttccacgccgccccccgtcct

SEQ ID NO: 24 - DNA, 25 bp, Artificial Sequence
  Description of Artificial Sequence Synthetic oligonucleotide
ttttttttttttttttttttttttt

SEQ ID NO: 25 - protein, 4 aa, Artificial Sequence
  Description of Artificial Sequence Synthetic peptide
GGGS

SEQ ID NO: 26 - DNA, 42 bp, Artificial Sequence
  Description of Artificial Sequence Synthetic oligonucleotide
  modified_base (21)..(21): inosine
aaaaaaaaaaaaaaaaaaaanttccakgccgccccccgtcct

SEQ ID NO: 27 - protein, 12 aa, Artificial Sequence
  Description of Artificial Sequence Synthetic peptide
  MOD_RES (3)..(10): Any amino acid
GSXXXXXXXXGG

SEQ ID NO: 28 - protein, 12 aa, Artificial Sequence
  Description of Artificial Sequence Synthetic peptide
GSDYKDDDDKGG

(19) United States
(12) Patent Application Publication
(10) Pub. No.: US 2024/0002838 A1
(43) Pub. Date: Jan. 4, 2024

(54) HIGH-SPEED PHOTO-CROSS-LINKING LINKER FOR MOLECULAR INTERACTION ANALYSIS AND IN VITRO SELECTION, AND IN VITRO SELECTION METHOD USING LINKER

(71) Applicant: Saitama University, Saitama city (JP)

(72) Inventors: Naoto Nemoto, Saitama City (JP); Yuki Mochizuki, Saitama City (JP); Shigefumi Kumachi, Saitama City (JP)

(73) Assignee: Saitama University, Saitama City (JP)

(21) Appl. No.: 18/368,817

(22) Filed: Sep. 15, 2023

Related U.S. Application Data
(62) Division of application No. 15/719,979, filed on Sep. 29, 2017.
Application No. 15/719,979 is a continuation of application No. PCT/JP2016/060611, filed on Mar. 31, 2016.

(30) Foreign Application Priority Data
Mar. 31, 2015 (JP) 2015-072810

(51) Int. Cl. C12N 15/10; C40B 30/04; C12N 15/09; G01N 33/68; C07K 14/00; C12N 9/00

(52) U.S. Cl. CPC C12N 15/1096; C40B 30/04; C12N 15/09; G01N 33/6845; C07K 14/003; C12N 9/93; C12N 15/101; C12Y 605/01001; C12N 2310/3513; C12N 2529/10

(57) ABSTRACT

Provided is a linker for both screening assessment of the candidate clones without using enzymes, and to provide an in vitro selection method using thereof. Also, provided is a high-speed photo-crosslinking linker for molecular interaction analysis and in vitro selection comprising a backbone and a side chain. The backbone comprises a solid-phase binding site located at the 5′ terminus for forming a bond with a solid-phase; a solid-phase cleavage site for releasing the entire solid-phase at the site; a side chain linking site for linking a side chain; a high-speed photo-crosslinking site for linking the backbone to mRNA having a sequence complementary thereof via photo-crosslinking; and a reverse transcription initiation region located adjacent to the side chain linking site at the 3′ terminus of the backbone. The side chain comprises a fluorescent label, a protein binding site located at the free terminus thereof; and a binding site with the backbone.

### CROSS REFERENCE TO RELATED APPLICATIONS

This application is a Divisional of U.S. patent application Ser. No. 15/719,979, filed on Sep. 29, 2017, which application is a Continuation of International PCT Patent Application No. PCT/JP/2016/060611, filed on Mar. 31, 2016, which application claims priority to Japanese Patent Application No. JP 2015-072810, filed on Mar. 31, 2015. The entire contents of these applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 3, 2021, is named 1451306_104US3_SL.txt and is 16,627 bytes in size.

### TECHNICAL FIELD

The present invention relates to a linker used in cDNA display method, which is a technique that connect genotype and phenotype, and the utilizing method by using thereof. Specifically, it relates to a novel puromycin linker used in both of the in vitro selection and affinity measurement, and the in vitro selection method by using thereof.

### BACKGROUND ART

Until present, each of pharmaceutical companies has developed antibody drugs. It is time consuming for production of the antibody drug in vivo, because it requires the steps to administrate antigen molecules to the animals in a couple of times to collect blood from them; then the collected blood is purified to obtain the antibody. Also, it is difficult to completely synthesize the antibody in vitro, because the molecular weight of the antibody is large, 150 KDa, even if IgG, the smallest antibody.

On the other hand, epitopes or peptide aptamers are synthesized, because their molecular weights are small.

When they are employed as the pharmaceutical agents, the organic synthesis thereof is required in GMP level; however, the peptide aptamer may be synthesized in such high level standard. Also, since the molecular weight of the peptide aptamer is small, they are easily modified chemically, and freely stabilized on tips and the like. Furthermore, they are highly stable compared to antibody so that there is merit that the peptide aptamer stabilized on the tips and the like may be stored at room temperature.

Such peptide aptamers may be subjected to be synthesize and be selected by using genotype-phenotype linking technology (hereinbelow, it is sometimes simply referred to as “linking technology”) after preparation of mRNA. Then, there are examples for the linking technology such as cDNA display method, phage display method, ribosome display method, mRNA method and the like. Among them, cDNA display method is the best for peptide aptamer screening, considering needs of automatically and high through put performance.

Until present, for cDNA display method, linkers shown in FIGS. 1A to 1D are proposed and used. FIG. 1A shows the liker comprising a solid phase binding site, T4 RNA ligase which binds mRNA and a backbone thereof by using T4 RNA ligase, the backbone having a reverse transcription primer region, a peptide binding site, fluorescent label, and a side chain bound to the backbone (see patent document #1, hereinbelow, it is referred to as “the prior art 1”).

Also, FIG. 1B shows the linker comprising the same structure other than its backbone is partially double strand to which restriction site for releasing the linker from a solid phase is incorporated (see patent document #1, hereinbelow, it is referred to as “the prior art 2”).

Also, FIG. 1C shows the linker of which structure is the same as that of the prior art 1 and additionally comprising both of the first and second cleavage sites (see the patent document 1, hereinbelow, it is referred to as “the prior art 3”). FIG. 1D shows the linker having two backbones ligated by solaren (see the patent document 2, hereinbelow, it is referred to as “the prior art 4”).

Another linker to which cnvK is incorporated in its backbone for cross linking mRNA and the backbone by using light (see the patent document 3, hereinbelow, it is referred to as “the prior art 5”).

At present, the first leading cause of death in Japanese is cancer (tumor), and its ratio among the causes of death is increasing year by year. When tumor is growing in a body, particular substances (tumor marker) with certain levels, for observing whether a person is affected by the tumor or not, and process after the tumor treatment, are detected in blood or urine. Therefore, it is known that the presence or absence of the tumor in the body, or tumor staging by determining types of the tumor markers, and there levels in the blood or urine.

Here, most of the tumor markers are carbohydrate antigens. When a normal cell becomes cancerous, chain length of the carbohydrate on the cell is changed by glycosyltransferase to cancer cell specific carbohydrate chain. Therefore, such carbohydrate chains are called tumor markers (hereinbelow, it is referred to as “carbohydrate tumor marker”) to identify the cancer cell (see non-patent document #5). Here, an antigenic determinant (hereinbelow, it is sometimes referred to as an “epitope”) has a general structure schematically shown in FIG. 2(A), and it is classified into type 1 carbohydrate, type 2 carbohydrate, scaffold carbohydrate, and core protein.

TABLE 1

| Locations of the carbohydrate antigens | | Names of the tumor markers |
|---|---|---|
| Carbohydrate | type 1 carbohydrate | CA19-9, CA50, Span-1, KMO-1, |
| antigens in a core | antigen | Dupan-2 |
| structure | type 2 carbohydrate | SLX, CSLEX |
| | antigen | |

| | |
|---|---|
| Carbohydrate antigens in the scaffolds | CA72-4, A546, STN |
| Carbohydrate antigens in the core protein | CA125, CA602, CA130 |

Here, it is known that those listed in the table 1 as both of the type 1 carbohydrate antigen and type 2 carbohydrate antigen. All of the carbohydrate length of them are elongated by the glycosyltransferase, and are longer than those on the normal cell. Also, the core protein means that the protein surrounding the vial nucleic acids.

FIG. 2(B) schematically shows the structure of CA19-9 (Carbohydrate Antigen 19-9), which is one of the carbohydrate antigen against the core structure as one example of such carbohydrate antigens. CA19-9 is composed of 5 monosaccharides having the molecular weight of 874, in which each monosaccharide is connected via glycoside bindings. In the figure, NeuNac means N-acetyl neuraminic acid, Gal means galactose, Fuc means fucose; GlcNAc means N-acetyl-D-glucosamine, respectively. It is known that GlcNAc included in CA19-9 as shown in FIG. 2(B) is included in a variety of carbohydrate antigens other than CA19-9 (see the non-patent document #5, it is referred to as the “prior art 6” hereinbelow.)

Also it is known that the antigen is composed of right chains and heavy chains so that the molecular weight of immunoglobulin (IgG) is large, 170 KDa. In contrast, the antigen derived from Camelidae genus lacks right chain and is composed of solely heavy chains; and therefore, it has small molecular weight, 12 KDa, high plasticity of the conformation, and excellent thermostability. Hereinbelow, the molecule has such a region, a domain, is sometimes referred to as “VHH”.

### PRIOR ART

### Patent Document

[Patent document No. 1] Patent No. 4318721
[Patent document No. 2] Patent No. 2013-39060
[Patent document No. 3] Patent No. WO2014/142020

### Non-Patent Document

[Non-Patent document No. 1] Nucleic Acid Research, 2009, 1-13dot: 10.1093/nar/gkp514
[Non-Patent document No. 2] Ueno, S., J Biotechnol. 162, 299-302 (2012)
[Non-Patent document No. 3] Nemoto N, et al., Anal. Chem. 86, 8535-8540 (2014)
[Non-Patent document No. 4] Small T, et al., Methods in Molecular Biology 911, 2012, pp 3-13
[Non-Patent document No. 5] Emi IKEBE, Journal of Bioscience and Bioengineering, Vol. 188, pp 92 to 94, 2014

### SUMMARY OF THE INVENTION

### Problems to be Solved by the Invention

The prior arts described above are excellent as linkers being employed in cDNA display method. However, it is assumed that mRNA is used in cDNA display, and it requires an enzyme, T4 RNA ligase, to bind a backbone of the linker and mRNA. On the other hand, it is impossible to remove RNase contaminant completely in a sample used in a common laboratory where E. coli is employed. Therefore, there is a problem that mRNA for the peptide aptamer synthesis is degraded in a test tube, it leads that the peptide aptamer by using the linker of the prior arts #1 to 4 is not successfully synthesized.

For example, the linker of the prior art #3 has 2 cleavage sites in the backbone for releasing the solid phase and the linker to recover the peptide displayed easily (see FIG. 1C). In this case, the release is conducted by using RNase I when rG is incorporated as the site, however, this case has the problem that used RNase I enzyme degrades mRNA. Therefore, it is excellent that the linker is modified to use another enzyme except RNase I, endonuclease V, is employed to release the linker from the solid phase by incorporating deoxy inosine (dI) at the cleavage site; thereby mRNA degradation was reduced.

However, every linker of the prior arts utilizes T4 RNA ligase enzyme to bind the backbone of the linker and mRNA so that it takes more than 30 minutes for bonding them.

As a method for shorten the binding time, the photo-cross-linking method by using irradiation of light having the wave length of 290 to 300 nm (it is sometimes referred to as “UV”) with the solaren for about 15 minutes is proposed. The photo-cross-linking method has a problem that thymidine dimers are formed, and they cause frequent mutation. Also, the method gives damages to mRNA, and causes unsuccessful reverse transcription and poor cDNA display efficiency. Further, position to which solaren is incorporated in the backbone is only at the 5′ terminal of the linker (see FIG. 1D); thereby it adds big limitation for design of the linkers.

Therefore, there are strong needs for the methods which do not use any enzymes nor give any damages to mRNA to enable to bind the linker and mRNA in short time, and no design limitation.

Alternatively, in cDNA display method, a selection process is repeated that a bound body which is composed of the linker, mRNA, cDNA and a peptide is formed and collected, and the displayed cDNA or the peptide is purified to specify its sequence, thereby constructing cDNA library or peptide library. However, the linker used in the selection process is not able to use in an experiment for analyzing intermolecular interaction. Namely, the linker was not able to both in vitro selection experiment for obtaining the candidate clone (screening) and the evaluation of the binding property of the clone obtained by the screening. Therefore, different linkers were used for the screening of the candidate clone and the evaluation test for the binding property of the clone; this caused problem that the work efficiency and cost thereof.

Accordingly, there are strong social needs for preparing the clone to be used both screening of the candidate clone and the evaluation of the binding property of the clone.

Furthermore, to specific detection of the target protein, it is preferable to use any antibodies such as immunoglobulins. However, there are problems such as the antibody being composed of the light chains and heavy chains has large molecular weight, it is irreversibly inactivated at the temperature of 70 degree centigrade or more thereby it is not able to be chemically synthesized. Also, there is another problem that sugar chains have wide variety, and their structures are complicated so that the antibody which recognizes the carbohydrate antigen is almost impossible.

On the other hand, the antigen derived from Camelids, for example, VHH, it is reversibly denatured by heat treatment at the temperature of 90 degree centigrade, and it shows the same level of the antigenicity as that before heat treatment when it is cooled to room temperature. However, there are problems that the antibody by Camelids has low production rate, and is purified through time-consuming process, and further the obtained antibody is not necessarily the interested one.

If the molecules that specifically recognize the carbohydrate antigens are obtained, it makes quick and proper judgement for the prophylaxis and treatment efficiency of cancer by utilizing the molecule as diagnostics. Then, such judgement is allowed, it makes proper treatment in early stage for a patient, even if the patient is affected the cancer, thereby being enabling to improve recovery ratio.

Accordingly, there is strong need to quickly and conveniently obtain the molecule having the epitope which enables to bind the antibody or the target molecule as the interest.

### Means for Solving the Problem

Under such circumstances, the present inventors studied hardly, and completed the present invention.

Namely, one feature of the present invention is a high-speed photo-cross-linking shared linker for in vitro selection and intermolecular interaction analysis, comprising a molecular backbone and a side chain: said molecular backbone comprising, a solid phase binding site having a predetermined nucleotide sequence and located at 5′ end thereof for forming a bond to bind to said solid phase; a solid phase cleavage site for cleaving said solid phase including said solid phase binding site; a side chain ligation site for ligating said side chain to said molecular backbone; a high-speed photo-cross-linking site locating between said side chain binding site for ligating mRNA having a complementary sequence with that of the molecular backbone by using photo-cross-linking to said molecular backbone; and a reverse transcription starting region adjacent to said side chain binding site and locating at 3′ end of the molecular backbone; said side chain comprising a fluorescent label, a protein binding site locating at a free end thereof, and a ligation formation site for being bound to said molecular backbone; and said side chain is ligated to said side chain ligation site at the ligation formation site in the molecular backbone.

Here, the solid phase cleavage site is preferably composed of a nucleotide selected from the group consisting of deoxy inosine, riboG and ribopyrimidine. Also, the high-speed photo-cross-linking site is preferably composed of cyano-vinyl carbazole compound, which is 3-cyano-vinyl carbazole.

The solid phase binding site is preferably composed of any one of the compound selected from the group consisting of biotin, streptavidin, alkyne compound, azide obtained through click chemistry, a compound having amino substitute, N-hydroxysuccinimide ester (NHS), the compound having SH substitute and Au, as well as poly A bounds thereto. The protein binding site is preferably composed of puromycin or derivative compounds thereof. As the derivative compounds, it is more preferable that the compound is any one selected from the group consisting of those such as 3′-N-aminoacylpuromycin (PANS-amino acid), nucleoside of 3′-N-aminoacyladenosine amino acid (AANS-amino acid), and the like may be used; PANS-Gly which has glycine as an amino moiety in PANS, PANS-Val which has valine as that, PANS-Ala which has alanine in that, a mixture of PANS amino acids, AANS-Gly which has glycine as the amino acid moiety in AANS, AANS-Val which has valine as the amino acid moiety in AANS, AANS-Ala which has alanine as the amino acid moiety in AANS, the mixture of AANS amino acids.

One of other feature of the present invention is a method for in vitro selection comprising the steps of: forming a complementary bond for binding the molecular backbone of the high-speed crosslinking shared linker for the in vitro selection and intermolecular interaction analysis to a desirable mRNA; photo-cross-linking by using irradiation of light having 300 to 400 nm wavelength for 0.5 to 5 minutes to both of said molecular backbone and mRNA which are mutually bound through a complementary bond; forming a fusion body being composed of mRNA-protein, wherein the protein is obtained through translation of mRNA bounds to the linker in cell-free translation system and said protein is bound to the linker; binding said fusion body to a solid phase; reverse-transcribing a mRNA included in the fusion body to obtain cDNA and to form a conjugate being composed of the fusion body and transcribed cDNA; and choosing desirable cDNA through cleaving the fusion body from the solid phase.

Here, said solid phase is preferably composed of a magnetic bead coated by either streptavidin or avidin. Also, said cleavage of the conjugate is preferably conducted by using any one of the enzyme selected from the group consisting of endonuclease V, RNase T1, and RNase A. The molecular backbone of the high-speed crosslinking shared linker preferably comprises a sequence for recognizing a carbohydrate antigen.

Further aspect of the present invention is a method for preparing a linker-protein for affinity measurement comprising the steps of: forming a complementary bond for binding the molecular backbone of the high-speed photo-cross-linking shared linker for the in vitro selection and intermolecular interaction analysis of the claim 1 to a desirable mRNA; photo-cross-linking by using irradiation of light having 300 to 400 nm wavelength for 0.5 to 5 minutes to both of said molecular backbone and mRNA which are mutually bound through a complementary binding; forming a fusion body being composed of mRNA-protein, wherein the protein is obtained through translation of mRNA bounds to the linker in cell-free translation system and said protein is bound to the linker; forming a fusion body being composed of the linker-protein by treatment of RNA digestion of the fusion body being composed of mRNA-protein; binding said fusion body being composed of mRNA-protein body to a solid phase; and purifying said fusion being composed of mRNA-protein eluted from the solid phase under a predetermined conditions, and then purifying hereof in an aqueous solution including 0 to 100 mM NaCl at room temperature.

Wherein the solid phase is preferably composed of a magnetic bead coated by either streptavidin or avidin. Also, the purification step is preferably conducted in an aqueous solution including 1 to 100 mM NaCl at room temperature.

Further aspect of the present invention is a linker-protein for affinity measurement prepared by using any one of the method according to the method described above.

### Advantageous Effect of the Present Invention

According to the present invention, the high-speed photo-cross-linking type linker for cDNA display which enables to greatly shorten binding time between the linker and mRNA is provided. By using the high-speed photo-cross-linking type linker for cDNA display, the method for in vitro selection which efficiently enables to choose a candidate clone is provided.

Furthermore, by using the high-speed photo-cross-linking type linker for cDNA display, a method for preparing a linker-protein for affinity measurement which enables to be used for evaluating the binding property of the candidate clones obtained by the method. Additionally, a linker-protein for affinity measurement produced by the method is provided.

### BRIEF DESCRIPTION OF DRAWINGS

FIG. 1A is a schematic figure showing a linker without a cleavage site (SBP) of the prior art 1.

FIG. 1B is the schematic figure showing the linker with the cleavage site in a double strand (restriction sites) of the prior art 2.

FIG. 1C is the schematic figure showing the linker with the cleavage sites in a single strand, which is used to be released from a solid phase of the prior art 3; wherein the first and the second cleavage sites in the linker are composed of ribosylguanosine (rG) or deoxy inosine (I).

FIG. 1D is the schematic figure showing the linker wherein two molecular backbones are cross-linked by using solaren of the prior art 4.

FIG. 2(A) schematically shows a structure of an epitope in a carbohydrate tumor marker, and FIG. 2(B) schematically shows the structure of CA19-9, one example of the epitope in the carbohydrate tumor marker.

FIG. 3A is the schematic figure showing the high-speed photo-cross-linking linker for dual use for molecular interaction analysis and in vitro selection.

FIG. 3B shows the structure of the linker of the present invention shown in FIG. 3A. FIG. 3B discloses SEQ ID NOS 26 and 21, respectively, in order of appearance.

FIG. 4 is the schematic figure showing that the linker of the present invention is used both in the molecular interaction analysis and in vitro selection.

FIG. 5 shows the structure of VHH library construct employed in the present invention (FIG. 5 discloses SEQ ID NOS: 10 and 25, respectively in order of appearance);

FIG. 6(A), FIG. 6(B) and FIG. 6(C) are graphs showing theoretical occurrence frequency of random amino acids, which are represented as a code for mixed nucleotide, on the SEQ ID NO: 6. FIG. 6(A) shows the case wherein the codon is nbn, FIG. 6(B) shows that wherein the codon is drb, and FIG. 6(C) shows that wherein the codon is yhm, respectively.

FIG. 7A is an image of gel electrophoresis showing cross-linking status under UV irradiation with UV with the linker of the present invention (+) or without it (−).

FIG. 7B is the image of gel electrophoresis showing the studied results for decomposition of the mRNA-linker conjugate (mRNA-linker fusant) depending on UV irradiation time (detected by FITC).

FIG. 7C is the image of gel electrophoresis showing the studied results for decomposition of the mRNA-linker conjugate (mRNA-linker fusant) depending on UV irradiation time (detected by SYBR Gold);

FIG. 7D is the image of gel electrophoresis showing the studied results for decomposition of the mRNA-linker conjugate (mRNA-linker fusant) depending on UV irradiation time, when the linker of the prior art 1 was used (detected by both of FITC or SYBR Gold).

FIG. 7E is the image of gel electrophoresis showing the studied results for decomposition of the mRNA-linker conjugate (mRNA-linker fusant) depending on UV irradiation time, when the linker of the present invention was used (detected by SYBR Gold).

FIG. 7F is the image of gel electrophoresis showing the studied results for decomposition of the mRNA-linker conjugate (mRNA-linker fusant) depending on UV irradiation dose, when the linker of the present invention was used (detected by SYBR Gold).

FIG. 7G is the image of gel electrophoresis showing the studied results for cDNA synthesis depending on UV irradiation time, when the linker of the present invention was used (detected by SYBR Gold).

FIG. 8(A) shows a chart showing the initial library (upper column) and FIG. 8(B) shows its distribution after termination of 3 rounds in in vitro selection (lower column). FIG. 8(A) discloses SEQ ID NO: 27 and FIG. 8(B) discloses SEQ ID NO: 28.

FIG. 9 is the chart showing the result for binding confirmation of competitive elution by using low concertation of GlcNAc non-binding protein.

FIG. 10 is the chart showing the result for binding confirmation of competitive elution by using high concertation of GlcNAc non-binding protein.

FIG. 11 is the gel electrophoresis results showing the difference of peptides included in washed solution and an eluate.

FIG. 12(A) and FIG. 12(B) are gel electrophoresis images showing products in the first round when the cross-linked peptide aptamer was prepared.

FIG. 13(A) and FIG. 13(B) are chromatograms of gel electrophoresis of the 6^{th} round products when the cross-linked peptide aptamer was prepared.

FIG. 14 is the chromatogram of gel electrophoresis showing the formation of mRNA-peptide conjugate, which is the conjugate of mRNA-linker and further linked peptide.

FIG. 15(A), FIG. 15(B) and FIG. 15(C) are chromatograms of gel electrophoresis showing the result confirming the progress of in vitro selection from the 1^{st} to 3^{rd} rounds.

FIG. 16(A) and FIG. 16(B) are chromatograms of gel electrophoresis showing the result confirming the progress of in vitro selection at the 4^{th} round.

FIG. 17(A) and FIG. 17(B) are chromatograms of gel electrophoresis of the result confirming the progress of in vitro selection at 5^{th} round.

FIG. 18(A) and FIG. 18(B) are chromatograms of gel electrophoresis of the result confirming the progress of in vitro selection at 6^{th} round.

FIG. 19 is the chromatogram of gel electrophoresis confirming the differences among the products depending on the presence or absence of cDNA display of VHH peptide.

FIG. 20 is a chromatogram of gel electrophoresis showing that the mRNA and the linker of the present invention are linked by photo-cross-linking.

FIG. 21A shows the nucleotide sequence and the restriction cites in the backbone of the linker of the prior art #2.

FIG. 21B shows that the linker of the prior art #2 is not cleaved by endonuclease.

FIG. 22 shows that the linker of the prior art #3 is cleaved by RNase, but the linker of the prior art #4 is not cleaved.

FIG. 23A shows segments for synthesizing the linker of prior art 5 (1).

FIG. 23B shows the segments for synthesizing the linker of prior art 5 (2).

FIG. 23C is the chromatogram of gel electrophoresis of products in the process of the linker of prior art 5 (1).

FIG. 23D is the chromatogram of gel electrophoresis of the products in the process of the linker of prior art 5 (2).

FIG. 23E is the chromatogram of gel electrophoresis of the products in the process of the linker of prior art 5 (3).

### MODE FOR CARRYING OUT THE INVENTION

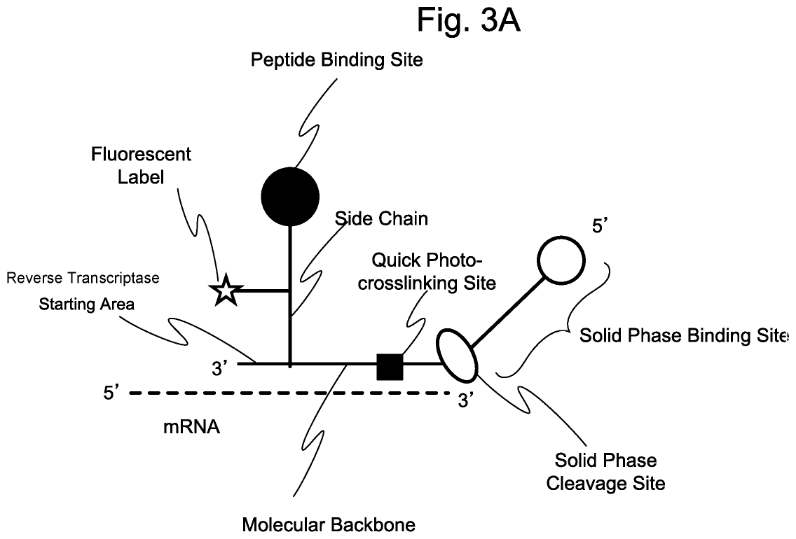

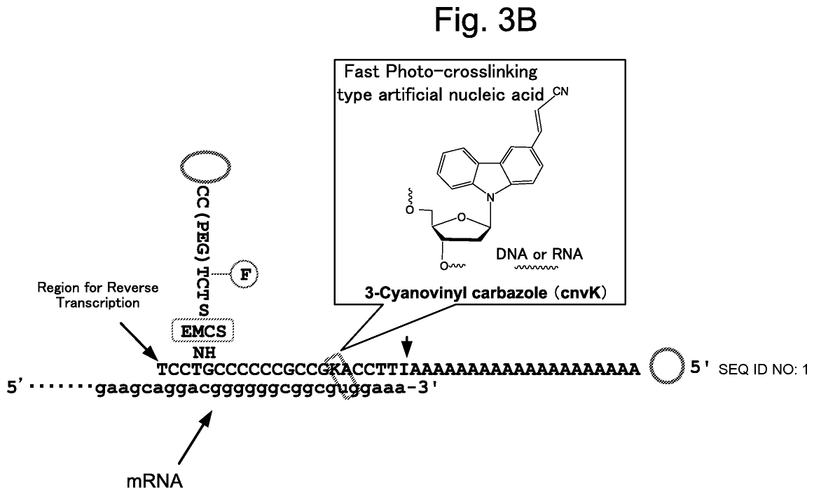

Hereinbelow, the present invention is explained in detail, referring FIGS. 3A to 5. As shown in the FIG. 3A, the present invention is a high-speed photo-cross-linking shared linker for cDNA display, comprising: (a) a molecular backbone and (b) a side chain. Said molecular backbone (a) comprising (a1) a solid phase binding site having a predetermined nucleotide sequence and locating at 5′ end thereof for forming a bonding to said solid phase; (a2) a solid phase cleavage site for separating said solid phase including said solid phase binding site; (a3) a side chain ligation site for ligating said side chain to said molecular backbone; (a4) a high-speed photo-cross-linking site locating between said side chain binding site and said solid phase cleavage site for binding said molecular backbone to mRNA having a complementary sequence with that of the molecular backbone by using photo-cross-linking; and (a5) a reverse transcription starting region adjacent to the sidechain binding site and locating at 3′ end of the molecular backbone. Said sidechain (b) comprises a (b1) fluorescent label, (b2) a protein binding site locating at a free end thereof, and (b3) a ligation formation site for being bound to the molecular backbone. Said side chain (b) is ligated to said side chain ligation side at the ligation formation site in the molecular backbone (a).

The solid phase binding site (a1) is preferably composed of any one of the compound selected from the group consisting of biotin, streptavidin, alkyne compound, azide obtained through click chemistry, a compound having amino substitute, N-hydroxysuccinimide ester (NHS), the compound having SH substitute and Au, as well as poly A, which is preferably composed of at least 10 adenine, because it enables to hold a suitable space between the solid phase and the linker, and is available for successful release of the linker from the solid phase described later. More preferably, poly A comprises about 20 adenines.

Also, the solid phase cleavage site is preferably composed of a nucleotide selected from the group consisting of deoxy inosine, riboG and ribopyrimidine, because of the following reasons. In order to release the conjugate of mRNA, cDNA and the linker of the present invention or mRNA, cDNA, a peptide and the linker of the present invention, hereinbelow, they are sometimes collectively referred to as the “conjugate”, any one of the enzyme selected from the group consisting of endonuclease V, RNase T1 and RNase A is used. By this, the solid phase is cleaved from the linker, including the solid phase binding site, if the solid phase cleavage site is composed of any one the nucleotides as mentioned above.

By this, the present linker enables to collect the conjugate (complex) of the RNA and thereof, that of protein having the amino acid sequence corresponding to cDNA/mRNA and thereof into the supernatant of the reaction mixture with no effects, even if these are included in the mixture.

Alternatively, the molecular backbone includes the side chain ligation site (a3) for ligating said side chain to said molecular backbone, and the side chain is explained later. Then, the high-speed photo-cross-linking site (a4), which ligates mRNA having complementary sequence to the molecular backbone by using photo-cross-linking, is placed so as to be located between the side chain ligation side and the solid phase cleavage site. The high-speed photo-cross-linking site is preferably composed of cyanovinyl carbazole compounds, because it does not cause decomposition of mRNA.

As the cyanovinyl carbazole compounds, there are mentioned 3-cyanovinyl carbazole and the like. When, 3-cyanovinyl carbazole (herein-below, it is referred to as “cnvK”) is used, mRNA to be subjected to screening and the molecular backbone of the linker were ligated by using UV with long wavelength in water during very short time. This means that the present invention does not need any enzymes such as T4 RNA ligase and the like, and also this enables to conduct the crosslinking reaction in water.

A buffer including metal ion such as Zn²⁺ is essential for the enzyme reaction for ligation, however, it is impossible to remove RNase which decompose RNA from the buffer. When T4 RNA ligase is used for ligation of mRNA and the molecular backbone, RNase is also activated under the condition that T4 RNA ligase is activated. Therefore, RNase decomposes mRNA so that it prevents cDNA synthesis sometimes. However, the high-speed photo-cross-linking site being composed of cnvK makes possible to conduct photo-cross-linking reaction without any enzymes in water, wherein the enzyme cannot function. Therefore, the decomposition of mRNA by RNase is prevented.

The light used for the crosslinking for the photo-cross-linking has rather long wavelength, 300 nm to 500 nm, as described later and its irradiation time is short. Therefore, it has advantages that there is no trouble that synthesized cDNA includes thymine dimer, and desirable peptides having the complementary sequence to mRNA used.

Next, the reverse transcription starting region (a5) for cDNA synthesis, wherein cDNA has the corresponding sequence to mRNA ligated to the linker, is formed so as to adjacent to the side chain binding site and located at the 3′ end of the molecular backbone.

The side chain (b) contained in the present linker comprises the fluorescent label (b1), a protein binding site (b2) located at the free end, ligation formation site (b3) to be ligated to the side chain ligation site in the molecular backbone. Wherein, as the fluorescent label (b1), for example, there is mentioned such as fluorescein, rhodamine, Cy dye, Alexa R Fluor and the like. More concretely, FITC is preferably used from the view point of cost.

The protein binding site (b2) located at the free end of the side chain is preferably composed of puromycin or the derivatives thereof. As the derivatives of puromycin, for example, there are mentioned such as 3′-N-aminoacyl puromycin (PANS-amino acids) and 3′-N-aminoacyl adenosine amino acid nucleoside (AANS-amino acid) and the like. More concretely, there are mentioned such as PANS-Gly of which amino acid portion of PANS is glycine, PANS-Val of which the portion is valine, PANS-Ala of which the portion is alanine, mixture of PANS amino acids, AANS-Gly of which amino acid portion of AANS is glycine, AANS-Val of which the portion is valine, AANS-Ala of which portion is alanine, the mixture of AANS amino acids, and the like.

Puromycin is preferably used, because it makes the synthesis of the linker easy, also the synthesized linker is handled without difficulty, and the cost thereof is low.

The ligation formation site in the side chain (b3) is preferably composed of divalent agent enables to make crosslinking between the amino acid and SH substitute. For example, N-(6-Maleimidocaproyloxy) succinimide (hereinbelow, it is sometimes referred to as “EMCS”) may be used. EMCS is preferably used, because its cost is low and it is handled without difficulty. The side chain (b) is ligated to the side chain ligation side (a3) in the molecular backbone at the ligation formation site (b3).

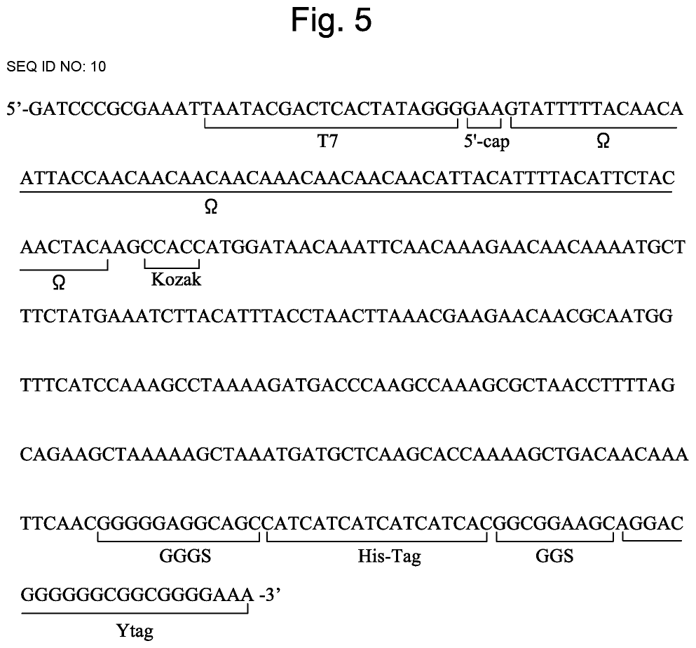

By employing such configuration, cDNA display linker of the present invention is bound to mRNA having the desirable sequence to form the conjugate including the peptide having the sequence complementary to that of mRNA. Then, the conjugate is stabilized on the solid phase to form another conjugate to which cDNA produced by using the reverse transcription of the peptide. After that, the solid phase is cleaved by using any one of the enzyme selected from the group consisting of endonuclease V, RNase T1, and RNase A to obtain cDNA display molecule. Namely, it may be used as the linker for cDNA display (FIG. 5).

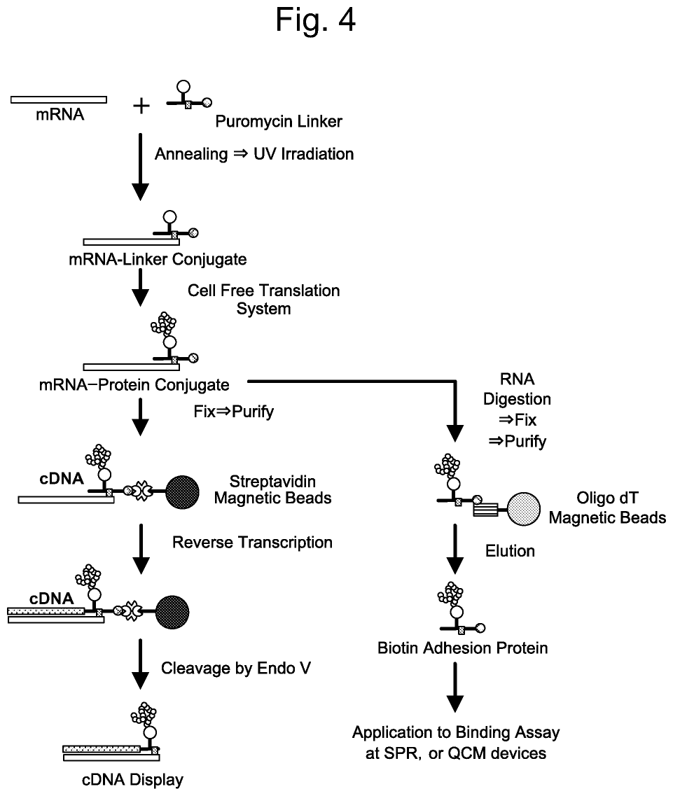

RNA in the mRNA—peptide—linker conjugate is digested by using the enzyme, and after that, for example, the digested conjugate is bound on the solid phase such as Oligo dT magnetic beads and the like at the solid phase binding site thereof and then eluted by using the elution buffer. By this, the protein-linker conjugate having the solid phase binging site is obtained; and the conjugate obtained is employed in the binding assay using SPR (surface plasmon resonance), QCM (quartz crystal microbalance) and the like (see FIG. 4).

The linker for cDNA display of the present invention as described above may be produces as follows. Firstly, the molecular backbone, which is sometimes referred to as “poly A+cnvK segment”, is designed so as that the cnvK is placed at the desirable position between the solid phase binding site and the side chain binding site, and the designed DNA is chemically synthesized according to the conventional method. Such chemical synthesis of the DNA chain may be outsourced to a company conducting such synthesis.

Such molecular backbone is designed so as to comprise, for example, the reverse transcriptase region, the side chain binding site, the high-speed cross-linking site, and the solid phase binding site as shown in FIG. 3B. The partial nucleotide sequence, from which modified region is omitted; of the molecular backbone shown in FIG. 3B is shown as the sequence (SEQ ID NO: 26 in the sequence listing) in below. In the following molecular backbone, Bio TEG is added at the 5′ terminal. Also, in the following nucleotide sequence, N represents inosine, X represents amino C6-dT.

### [SEQ ID NO: 1]

5′AAAAAAAAAAAAAAAAAAAASTTCCAGCCGCCCCCCGVCCT 3′

The side chain of the present linker (puromycin—segment) is also designed so as to have the desirable sequence, and the DNA chain is chemically synthesized as the same as that of the Poly A+cnvK segment according to the conventional method. Such chemical synthesis of the DNA chain may be outsourced to a company conducting such synthesis.

Such side chain may be designed, for example, so as to comprise the ligation site shown in FIG. 3B, the fluorescent molecule, and the protein binding site. In the side chain shown in FIG. 3B, the nucleotide sequence from which modified region I omitted is illustrated by an example as follows. Here, the solid phase cleavage site is preferably composed of the nucleotide selected from the group consisting of deoxy inosine, riboG and ribopyrimidine; because it makes the linker specifically cleaved from the solid phase later. In the following side chain, P which is located at the free end is puromycin as the protein binding site. Also, in the following nucleotide sequence, (5S) represents 5′ Thiol C6, F represents FITC-dT, and Z represents Spacer 18, respectively.

5′ (5S)TCTFZZCCP

For example, EMCS (Dojindo Molecular Technologies, Inc.) is added so as that its final concentration becomes 15 to 18 mM to 0.1 to 0.3 M sodium phosphate solution (pH 7.0 to 7.4) including 10 to 20 nmol (final conc. 100 to 200 μM) of the molecular backbone having such sequence. Then, the buffer is incubated at 37° C. for 20 to 40 minutes, and then they are precipitated by using ethanol precipitation. Preferably, EMCS is added to about 0.2 M sodium phosphate solution (about pH 7.2) including about 15 nmol of the molecular backbone as described above (final conc. is about 150 M) so as that the final concentration of EMCS becomes about 16.7 mM, and incubated at 37° C. for about 30 minutes. Then, the ethanol precipitation is carried out by using, for example, Quick-Precip Plus Solution (Edge BioSystems).

Next, 30 to 45 nmol of the side chain is dissolved in the 0.8 to 1.5 M sodium hydrogen phosphate solution including 40 to 60 mM DTT so as that the final concentration of the side chain becomes about 400 to 430 μM. Then, the solution is stirred by using a shaker at room temperature for about 0.75 to 1.5 hour. Subsequently, the solution is subjected to buffer exchange. Preferably, about 37.5 nmol of the side chain is dissolved in about 1 M sodium hydrogen phosphate solution including about 50 mM DTT so as that the final concentration of the side chain becomes about 417 μM. Then, the solution is stirred by using a shaker at room temperature for about 1 hour. Subsequently, the solution is exchanged to about 0.1 M sodium phosphate buffer including about 0.15 M NaCl (about pH 7.0) by using NAP5 column and the like.

Next, the solution including reduced side chain whish was subjected to the buffer exchange as described above is mixed with the ethanol precipitate with EMCS modification and stood overnight at 2 to 6° C. Then, DTT is added to the solution so as that the final concentration of it becomes 40 to 60 mM, and stirred at room temperature for 15 to 60 minutes. After that, the solution is subjected to ethanol precipitation, and the obtained ethanol precipitate is dissolved in 50 to 200 μM Nuclease free water for purification. Preferably, the buffer-exchanged solution including the reduced side chain of which is that described above is mixed with the ethanol precipitate of the molecular backbone which was modified with EMCS as described above, and stood at about 4° C. overnight.

Then, DTT is added to the reaction mixture so as that the final concentration of DTT becomes about 50 mM, and stirred at room temperature for about 30 minutes. Then, the mixture is subjected to ethanol precipitation by using Quick-Precip Plus Solution (Edge BioSystems). The obtained ethanol precipitate is dissolved in about 100 μL of Nuclease-free water, and then it is subjected HPLC purification by using C18 column with gradient elution, for example, under the following conditions. By this, the present linker is obtained.

The elution buffer for the gradient elution is composed of, for example, A solution comprising 0.05 to 0.2 M trimethyl ammonium acetate (in ultra-pure water) and B solution comprising 75 to 85% of acetonitrile. The ratio of A solution in the elution buffer at the start may be decreased about 20% during 40 to 50 minutes from the start. Flow rate may be 0.5 to 1.5 ml/minute, and the fraction may be 0.5 to 1.5 mL. Preferably, A solution is about 0.1 M trimethyl ammonium acetate (in ultra-pure water), and B solution is about 80% of acetonitrile; then, the ratio of the A solution (about 85%) is decreased to about 65% during 40 to 50 minutes; the flow rate is about 1.0 mL/minute, the fraction is about 1.0 mL.

The components in the fractions are investigated by using both of fluorescence and UV absorption (for example, 280 nm). Then, the fractions showing peaks by using both of detection means are collected to evaporate the solvent by using a vacuum evaporator, and then it is subjected to ethanol precipitate. The ethanol precipitate is dissolved in Nuclease-free water to produce the present linker. The obtained present linker is stored at about −20° C. For example, when the fractions obtained between 30 to 32 minutes have the peaks detected by both of the fluorescence and UV, these fractions are collected and the solvent of the fractions are evaporated by using the vacuum evaporator. Then, for example, the evaporated solution is subjected to ethanol precipitation by using Quick-Precip Plus Solution, and the precipitates may be dissolved in Nuclease-free water to be stored at about −20° C.

### (In Vitro Selection Methods)

The library DNA having desirable sequences and DNA coded by FLAG sequence are mixed in the desirable molar ratio, for example, 25,000 to 100,000:1 to prepare DNA mixture, and take a portion to conduct transcription. For example, 250 to 1,000 ng thereof is subjected to the transcription in 10 to 20 μL scale by using a kit such as RiboMAX Large Scale RNA Production Systems-T7 and the like. As the sequence for the transcription, for example, the following one (SEQ ID NO: 2 in the sequence listing) may be employed. In the following sequence, N represents optionally A, T, G, and C, and K represents G or T.

[SEQ ID NO: 2: Amino acid random library
DNA sequence]
GATCCCGCGAAATTAATACGACTCACTATAGGGGAAGTATTTTTACAA
CAATTACCAACAACAACAACAAACAACAACAACATTACATTTTACATT
CTACAACTACAAGCCACCATGGGCAGCNNKNNKNNKNNKNNKNNKNNK
NNKGGAGGTGGAATTAAAAACATGTGCAATTTGAACCCACTTTTAAAA
AAGTGGCTAAATGATGCAAAGGGGGGAGGCAGCCATCATCATCATCAT
CACGGCGGAAGCAGGACGGGGGGCGGCGTGGAAA

The DNA mixture is incubated at desirable temperature for desirable time period, for example, at about 37° C. for 3 to 5 hours, and then DNase is added at the desirable amount. After that, the mixture is further incubated at the desirable temperature for the desirable time period, for example, at about 37° C. for 5 to 15 minutes. Obtained mRNA is then purified. Preferably, the DNA mixture is incubated about 37° C. for 4 hours, and about 0.5 μL of DNase being attached to the kit (for example, RQ1 Dnase, Promega) is added and further incubated at about 37° C. for 10 minutes. The obtained mRNA is purified by using, for example, After Tri-Reagent RNA Clean-Up Kit (Favogen Biotech Corp.).

As the same as those described above, photo-cross-linking of mRNA and the present linker (poly A+cnvK) is conducted by radiation of UV with long wavelength for 0.5 to 5 minutes. Obtained biotin-cnvK linker-mRNA conjugate (hereinbelow, it is sometimes referred to as “mRNA-linker conjugate”.) is subjected to the translation at the desirable temperature for the desirable time period at the same scale as described above by using cell-free translation system. For example, the translation is conducted by using 10 to 20 μL of mRNA-linker conjugate for translation at 100 to 150 μL scales, for example, it is conducted at about 30° C. for about 15 minutes by using the cell free system such as rabbit reticulocyte lysate and the like. Then, both of MgCl₂ and KCl are respectively added at the desirable concentrations to obtain the mRNA-peptide conjugate, which is the conjugate of mRNA-linker conjugate with the peptide. For example, both of MgCl₂ and KCl are added to the system so as that their concentrations are respectively 75 mM or 900 mM, and then incubated at about 37° C. for 1 hour to obtain the translation solution including mRNA-peptide conjugates.

To the translation solution obtained as described above, 0.25 to 1M of EDTA (pH 7.8 to 8.2) is added at the desirable concentration and incubated at the desirable temperature to remove ribosome bound to the mRNA-peptide conjugate. For example, about 0.5 M of EDTA (pH about 8.0) is added to the translation solution so as that the final concentration of EDTA becomes about 83 mM and incubated for about 5 minutes.

Subsequently, equal volume of the desirable binding buffer is added to the translation solution treated as mentioned above. Then, the translation solution is mixed with the desirable amount of magnetic beads with streptavidin washed with the binding buffer, and then they are stirred at the desirable temperature at the desirable time period. For example, the equal volume of 2× binding buffer (containing about 20 mM Tris-HCl (pH about 7.5), about 2 M of NaCl, about 2 mM EDTA, and about 0.2% of Tween-20) for SA is added, and mixed with the magnetic beads which are washed with 1× binding buffer for SA, for example, 100 to 200 μL of DYNABEADS MYONE (magenetic beads) C1 streptavidin, and then stirred with about 20 to 30° C. for 20 to 40 minutes.

The magnetic beads are washed with the desirable volume of 1× binding buffer for SA at desirable times, and then they are subjected to reverse transcription. For example, the beads are washed with about 200 μL of 1× binding buffer for SA at 2 to 4 times. Then, for example, they are subjected to the reverse transcription according to the protocol attached with REVERTRA ACE (reverse transcriptase) (a registered trademark) by using about 100 μL of reverse transcription reaction mixture, and stirred at about 42° C. for about 15 minutes to prepare mRNA/cDNA-peptide conjugate (it is referred to as “cDNA display”).

The magnetic beads are washed with the desirable volume of 1×NE buffer. Then, 1×NE buffer containing any one of the enzyme selected from the group consisting of endonuclease V, RNase T1, and RNase A, is added to the beads and stirred at the desirable temperature for the desirable time period. Next, the desirable volume of 2× His-tag washing buffer is added, and then the supernatant is recovered. For example, DYNABEADS MYONE (magenetic beads) C1 streptavidin is washed with about 150 μL of 1×NE buffer, and then about 75 μL of 1×NE buffer containing about 10 U of Endonuclease V is added, and stirred at about 37° C. for about 1 hour. Next, about 75 μL of 2× His-tag washing buffer (40 mM sodium phosphate buffer (pH about 7.4) containing about 1 M of NaCl, about 0.1% of Tween-20) is added, and the supernatant is recovered.

Next, the recovered supernatant is mixed with the magnetic beads, for example, His Mag Sepharose Ni washed with 1× His-tag washing buffer. Then, the mixture is stirred at the desirable temperature for the desirable time period by using a mixer. The beads are washed with 1× His-tag washing buffer for desirable times, and then, the desirable selection buffer is added and stirred at the desirable temperature for the desirable time period by using the mixer to recover the supernatant. For example, about 150 μL of the recovered supernatant and about 20 μL of His Mag Sepharose Ni (GE Health Care, already washed with 1× His-tag washing buffer) are mixed, and stirred at room temperature for about 1 hour by using the mixer such as inteli mixer RM-2M (Toho K. K.).

The beads are washed with about 100 μL of 1× His-tag washing buffer for 1 to 3 times, and then about 30 μL of selection buffer enriched with EDTA concentration (about 50 mM tris-HCl buffer (pH about 7.4) containing about 1 M of NaCl, about 10 mM imidazole, about 5 mM EDTA and about 0.1% of Tween-20) is added, and then they are stirred at room temperature for about 10 minutes by using the mixer to recover the supernatant. As described above, the linker of the present invention is produced.

Desirable of amount of the obtained linker is taken out. For example, the desirable column is filled with anti-FLAG M2 affinity gel, and washed with the desirable amount of the selection buffer. Then, the desirable amount of the supernatant is leaded on the column; and stirred at room temperature for about 1 hour by using a rotator and the like. The column is washed with the desirable amount of the selection buffer for several times. For example, the desirable concentration of FLAG peptide (Sigma-Aldrich Japan Co. LLC.) is added, and stirred at room temperature for about 15 minutes by using the rotator to elute mRNA/cDNA-peptide conjugate competitively.

For example, a proper column such as MicroSpin Empty Columns (GE Health Care) is filled with about 25 to 100 μL of anti-FLAG M2 affinity gel (about 40 to 60% suspension), and then washed with about 150 to 250 μL of the selection buffer for 2 to 4 times. Then, about 50 to 150 μL is taken out from the supernatant, and loaded on the column, and then stirred at room temperature for about 1 hour by using the rotator. The column is washed with about 150 to 250 μL of the selection buffer for 3 to 5 times, and then about 50 to 150 μL of 3×FLAG peptide (50 to 150 ng/μL, Sigma-Aldrich Japan Co. LLC.) is added, and stirred at room temperature for about 15 minutes by using the rotator. By this, mRNA/cDNA-peptide conjugate bound to anti-FLAG M2 affinity gel is competitively eluted.

Elution buffer in the column is recovered by centrifugation, and subjected to ethanol precipitation, and then dissolved into the desirable volume of nuclease free water (Nuclease-free water). The ethanol precipitate is added to about 150 to 250 μL of PCR solution, and conducted PCT. For example, the elution buffer recovered by the centrifugation is ethanol precipitated by using Quick-Precip Plus Solution and the like, and then the precipitate is dissolved in about 15 μL of Nuclease-free water. The ethanol precipitate is added to about 150 to 250 μL of PCR solution (1× PrimeSTAR buffer (with Mg²⁺) containing about 0.2 mM dNTPs, about 0.4 μM of T7Ω new, about 4 μM of New Ytag for cnvK, about 0.02 U/μL of PrimeSTAR HS DNA polymerase), PCR is conducted as follows. Sequences of 2 peptides shown as the examples, T7Ω new and NewYtag for cnvK (SEQ ID NO: 3 and 4 in the sequence listing) are shown in below.

[SEQ ID NO: 3: T7Ω new sequence]
5′-GATCCCGCGAAATTAATACGACTCACTATAGGGGAAG
TATTTTTACAACAATTACCAACA-3′
[SEQ ID NO: 4: NewYtag of cnvK sequence]
5′-TTTCCACGCCGCCCCCCGTCCTGCTTCCGCCGTGATGAT-3′

Next, PCR is conducted by using the steps of, for example, (a1) 98° C. for 1 minute, (b1) 98° C. for 15 seconds, (c1) 68° C. for 30 seconds, (d1) 68° C. for 1 minutes, and (b1) and (c1) are conducted 25 cycles. Obtained PCR products are subjected to SDS gel electrophoresis to excise full construct DNA for purification according to the conventional method. Purified full construct DNA is added to the PCR solution, and then dispensed at the desirable volume, double strand DNA is obtained by PCR.

The PCR product obtained through PCR as described above is, for example, electrophoresed in 8 M urea denaturing 6% PAGE, the full construct DNA (260 to 300 mer) is excised and purified according to the convention method. The purified full construct DNA is added to about 150 to 250 μL of PCR solution (1× PrimeSTAR buffer (with Mg²⁺) containing about 0.2 mM dNTPs, about 0.4 μM of Newleft, about 0.4 M of NewYtag for cnvK, about 0.02 U/μL of PrimeSTAR HS DNA polymerase), and then the mixture is dispensed about 25 to 75 μL each and conducted PCR. By this, the double strand DNA is obtained. The sequence of Newleft shown (SEQ ID NO: 5 in sequence listing) as the peptide example to be used here is shown in below.

[SEQ ID NO: 5: Newleft sequence]
GATCCCGCGAAATTAATACGACTCACTATAGGG

PCR may be conducted by using the steps of, for example, (a2) 98° C. for 1 minute, (b2) 98° C. for 10 seconds, (c2) 68° C. for 30 seconds, (d2) 68° C. for 1 minute, and steps (b2) and (c2) are conducted 5 cycles. PCR solution conducted as describe above are purified as a whole, and to use in the next round. The procedure described above, from the transpiration of the library DNA to affinity selection, is repeated for the desirable rounds, for example, 3 rounds in total. Then the full construct DNA obtained is subjected to direct sequencing to obtain the linker of the present invention displaying FLAG-DNA with the unified sequence.

Next, the linker of the present invention used for the binding assay and the like is explained. The desirable protein DNA, of which sequence is known, is used, and obtained mRNA-protein-linker conjugate of which mRNA has a complementary sequence to the nucleotide sequence of the protein according to the same procedure described above. Here, as the protein having the known sequence, for example, B domain of A protein (hereinbelow, it is referred to as “BDA”. SEQ ID NO: 6 in the sequence listing) may be used.

[SEQ ID NO: 6: DNA sequence of BDA]
GATCCCGCGAAATTAATACGACTCACTATAGGGGAAGTAT
TTTTACAACAATTACCAACAACAACAACAAACAACAACAA
CATTACATTTTACATTCTACAACTACAAGCCACCATGGAT
AACAAATTCAACAAAGAACAACAAAATGCTTTCTATGAAA
TCTTACATTTACCTAACTTAAACGAAGAACAACGCAATGG
TTTCATCCAAAGCCTAAAAGATGACCCAAGCCAAAGCGCT
AACCTTTTAGCAGAAGCTAAAAAGCTAAATGATGCTCAAG
CACCAAAAGCTGACAACAAATTCAACGGGGGAGGCAGCCA
TCATCATCATCATCACGGCGGAAGCAGGACGGGGGGCGGC
GTGGAAA

Next, the desirable buffer and RNase H are added to the translation product containing the mRNA-protein-linker conjugate, and incubated at the desirable temperature for the desirable time period to decompose mRNA. The equal volume of the desirable binding solution to the reaction solution is added, and the desirable magnetic beads washed with the binding buffer are mixed; and then they are stirred at room temperature for about 15 to 45 minutes by using the rotator.

For example, about 1/9 volume of 10×NE buffer 2 and about 20 U of RNase H (Takara Bio) are added to the translation product including mRNA-peptide conjugate, and incubated at about 37° C. for 45 to 75 minutes to decompose mRNA. Next, for example, the reaction mixture is added to the equal volume of 2× binding buffer for oligo dT (20 mM Tris-HCl (pH about 7.5) containing about 1 M of LiCl, about 2 mM EDTA, and about 0.1% of Tween-20), and about 50 to 150 μL of magnetic beads, for example, DYNABEADS (magenetic beads) Oligo (dT)25 (SEQ ID NO: 24) (Thermo Fisher Scientific, already washed with 2× binding buffer for oligo dT), and stirred at room temperature for about 30 minutes with the rotator and the like.

The magnetic beads are washed with the desirable volume of the desirable buffer, and then the desirable volume of ultra-pure water is added and incubated to elute biotin adhesion protein. To biotin adhesion protein thus obtained, the equal volume of the desirable washing buffer thereof is added. Then, the desirable amount of the magnetic beads such as His Mag Sepharose Ni and the like are mixed and stirred at the desirable temperature for desirable time period.

For example, DYNABEADS (magenetic beads) Oligo (dT)25 (SEQ ID NO: 24) is used as the magnetic beads, they are washed with about 150 to 250 μL of 1× binding buffer for oligo dT for multiple times. After that, 200 to 250 μL of ultra-pure water is added, and incubated at 37° C. for 5 to 15 minutes to the linker binding to the desirable protein such as biotin adhesion BDA and the like. To the biotin adhesion BDA solution thus obtained, for example, the equal volume of the 2× His-tag washing buffer is added, and mixed with the volume of about 15 to 50 μL of the magnetic beads such as His Mag Sepharose Ni and the like (already washed with 1× His-tag washing buffer), and stirred at room temperature for 30 to 50 minutes by using the rotator and the like.

Next, they are washed with the desirable volume of 1× His-tag washing buffer. After that, the desirable elution buffer is added and stirred at room temperature for the desirable time period to elute the biotin adhesion protein. Then, the DNA sequence of the obtained biotin adhesion protein is determined.

For example, they are washed with about 50 to 150 μL of 1× His-tag washing solution for multiple times, and then, 50 to 60 μL of His-tag elusion buffer (20 mM sodium phosphate buffer (pH about 7.4) containing about 0.5 M of NaCl, about 250 mM imidazole, about 0.05% of Tween-20) is added, and then they are stirred at room temperature for 10 to 20 minutes by using the inteli mixer RM-2M to elute biotin adhesion protein to sequence DNA of the obtained biotin adhesion BDA.

After that, affinity of the obtained biotin adhesion BDA is determined by using Biacore X100 (GE Health Care) and the like, and rate at the association (kₐ) and the dissociation (k_{d}), and the affinity (K_{D}=k_{d}/kₐ) are obtained by using Biacore J software utilizing 1:1 Langmuir binding model (A+B=AB).

The dual use linker of the present invention is used for studying both of in vitro selection and intermolecular coupling.

A cross-linked peptide aptamer for in vitro selection by using cnvK linker may be prepared as follows. In the preparation for such peptide aptamer, antibodies from Camelids, for example, VHH, is preferably used, because they have excellent refolding properties and relatively long sequence of CDR3.

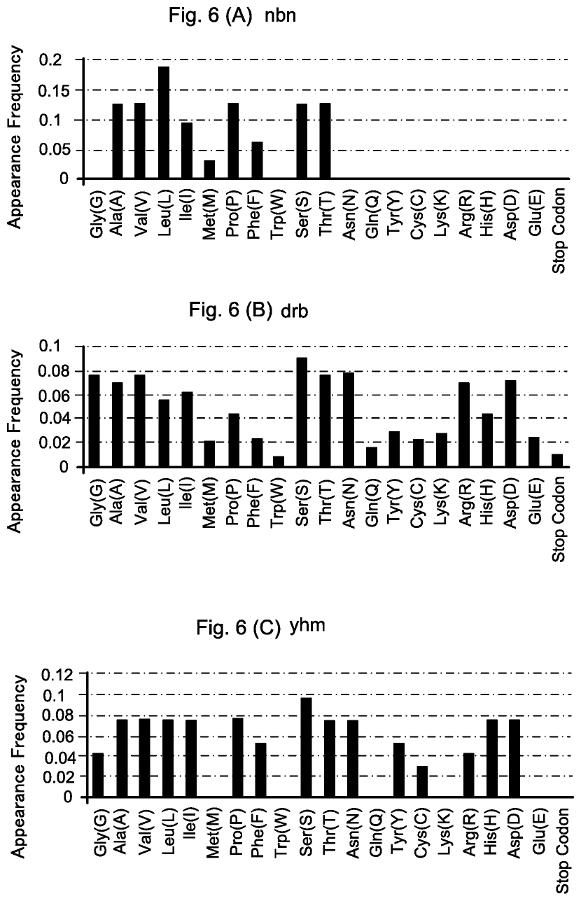

For example, according to the conventional method, VHH is ligated to the desirable sequence to structure VHH library construct to use it as a template DNA. As the desirable sequences, there are mentioned such as T7 region, Kozak region, 5′ cap region, Ω region, His-Tag, Y-tag, NewY tag, the region to be hybridized to the linker, and the like. As the construct comprising such sequences, for example, DNA sequence shown as SEQ ID NO: 7 in sequence listing and the like is preferably used. In the following SEQ ID NO: 7, n, b, d, r, y, h, and m respectively represent missed nucleotide, and their ratio is shown in below. The frequency appearance of each of the nucleotide is shown in table 2. Also, the theoretical appearance frequency of respective amino acid in each codon, which are shown as nbn, drb, and yhm are shown in FIG. 6(A) to (C).

(SEQ ID NO: 7)
5′-GATCCCGCGAAATTAATACGACTCACTATAGGGGAAG
TATTTTTACAACAATTACCAACAACAACAACAAACAACAA
CAACATTACATTTTACATTCTACAACTACAAGCCACCATG
GGCGAGGTGCAGCTGGTGGAGAGCGGAGGAGGATCCGTGC
AGGCTGGAGGAAGCCTGCGCCTGAGCTGCGCTGCTAGCGG
AnbnnbndrbyhmyhmyhmdrbnbnnbnTGGTTCCGCCAG
GCTCCTGGAAAGGAGCGCGAGGGAGTGnbnnbndrbyhmy
hmyhmyhmdrbACCTACTACGCTGACAGCGTGAAGGGACG
CTTCACCATCAGCCAGGACAACGCCAAGAACACCGTGTAC
CTGCAGATGAACAGCCTGAAGCCTGAGGACACCGCTATCT
ACTACTGCGCTGCTdrbdrbyhmyhmyhmyhmyhmyhmyh
myhmyhmyhmdrbTACTGGGGACAGGGAACCCAGGTGACC
GTGGGAGGAGGCAGCCATCATCATCATCATCACGGCGGAA
GCAGGACGGGGGGCGGCGGGGAAA-3′

TABLE 2

| | Appearance frequency of the nucleotide (%) | | | |
|---|---|---|---|---|
| Symbol | T | C | A | G |
| n | 25 | 25 | 25 | 25 |
| b | 50 | 50 | 0 | 0 |
| d | 19 | 27 | 27 | 27 |
| e | 28 | 28 | 28 | 16 |
| y | 13 | 20 | 35 | 32 |
| h | 24 | 22 | 30 | 24 |
| m | 37 | 37 | 0 | 26 |

After structuring of the construct including these sequences, they are subjected to PCR under the desirable conditions to amplify the construct. For example, the desirable amount of 5× Prime STAR Buffer respectively, dNTP mixture, primers, Prime STAR HS DNA polymerase are mixed, and VHH construct is added into them, and prepared, for example, desirable amount of PCR solution by using ultra-pure water.

Here, as the primers, there are mentioned such as T7 omega new (60 mer)(SEQ ID NO: 3 in sequence listing), NewYtag_for_PolyA&cnvK-Lin (22 mer) (SEQ ID NO: 23 in sequence listing), QRT-Lnew (32 mer) (SEQ ID NO: 8 in sequence listing), New left (33 mer) (SEQ ID NO: 5 in sequence listing), and NewYtag (22 mer) (SEQ ID NO: 9 in sequence listing), and the like.

PCR solution having the composition described above is prepared, and it is used for amplification by using the PCR program comprising, for example, at about 98° C. for 1.5 to 2.5 minutes, 4 to 6 cycles of (about 98° C. for 5 to 15 seconds, about 98° C. for about 1 second, about 65 to 70° C. for about 30 seconds to 50 seconds), and at about 70 to 75° C. for 1.5 to 2.5 minutes.

NewYtag_for_PolyA&cnvK-Lin (22 mer)
(SEQ ID NO: 23)
5′-TTTCCACGCCGCCCCCCGTCCT-3′
ΩRT-Lnew (32 mer)
(SEQ ID NO: 8)
5′-GGGGAAGTATTTTTACAACAATTACCAACAAC-3′

Here, diversity of the first solution (original solution) including VHH construct library is preferably about 2.5 to 5×10¹¹ molecules/μL, because a variety of VHH construct contained in the library is used as the material.

In order to select PCR products having the desirable chain length, the PCR products obtained by using PCR as described above is purified according to the conventional method, and short length DNAs are preferably removed. For such purification, any kit such as PCR Clean-Up MiniKit (Favorgen) and the like may be used. Then, PCR is conducted by changing the primers depending on the necessity, thereby obtaining PCR products having the desirable chain lengths. For example, among the primers, when New left is used, which is used instead of T7 omega new, and conduct PCR, and the desirable PCR products may be obtained by using the similar program described above.

Next, the obtained PCR products are transcribed to mRNA. Such transcription is conducted by using, for example, commercially available ones such as RiboMAX™ Large Scale RNA production Systems (Promega), or the desirable transcription buffer. As the transcription buffers, for example, T7 Transcription 5× Buffer, rNTPs, or the buffer prepared by using Enzyme Mix, a solution comprising template DNA dissolved in RNase-Free water.

The transcription buffer is incubated, for example, at about 36 to 38° C. for the desirable time period, for example 2.5 to 3.5 hours; then RNase-Free DNase is added, and incubated, for example, about 36 to 38° C. for the desirable time period, for example, 10 to 20 minutes to obtain mRNA. As RNase-Free DNase, for example, the commercially available ones such as RQ1 Rnase-Free DNAse (PROMEGA) and the like may be used.

After that, RNA is purified. For RNA purification, for example, After Tri-Reagent RNA Clean-up Kit (Favogen Biotech Corp.) may be used. It is preferable to adjust the amount of DNA as the template so as to obtain desirable RNA, and to obtain mRNA as used for photo-cross-linking with the linker.

Then, the purified mRNA and Biotin-cnvK linker thus produced are ligated by photo-cross-linking. Here, the photo-cross-linking buffer comprising the purified mRNA, Biotin-cnvK linker, about 0.75 to 1.5 M NaCl and about 0.2 to 0.3 M Tris-HCl is heated, for example, at about 88 to 92° C. for about 1 to 3 minutes, and then it is cooled to 65 to 75° C. within about 1 minute. After that, it is heated at about 68 to 72° C. for about 0.5 to 2 minutes; and then it is cooled to about 20 to 30° C. within about 15 minutes for annealing. Here, it is preferable that the molar ratio of mRNA and Biotin-cnvK linker in the photo-cross-linking buffer is almost equal in the viewpoint of crosslinking efficiency.

After the completion of the annealing, the UV having the long wavelength is irradiated at the desirable amount to the buffer for photo-cross-linking. For example, the commercially available device such as CL-1000 Ultraviolet Crosslinker (UVP) and the like is used to irradiate UV light having the wave length from 360 to 370 nm under the condition of the range from 350 to 450 mJ/cm² for cross-linkage to obtain mRNA-linker conjugate.

Subsequently, the mRNA-linker conjugate obtained as described above is translated in a cell free system to form mRNA-peptide conjugate. As the buffer to be used in the translation, for example, the solution containing Translation Mix, the mRNA-linker conjugate, Retic Lysate, and RNase inhibitor is prepared, and finally adjusted to the desirable volume by using ultra-pure water.

Cell free translation may be carried out by treating the tube containing the translation buffer as follows. For example, the tube is incubated at about 25 to 35° C. for about 15 to 25 minutes, then, both of about 10 to 15 μL of about 2.5 to 3.5 M of KCl and about 2 to 4 μL of 0.5 to 1.5 M of MgCl₂ are added, and further incubated at about 36 to 38° C. for about 45 to 75 minutes. Then, about 5 to 15 μL of ethylenediamine tetra-acetic acid (about pH 7.5 to 8.5) is added and incubated at about 36 to 38° C. for about 5 to 15 minutes. Then, about 40 to 80 μL of the binding buffer is added to obtain mRNA-peptide conjugate, wherein the peptide synthesized in the cell free system is binding to the mRNA conjugate described above.

Next, mRNA—peptide conjugate obtained as described above is bound onto the magnetic beads. As such magnetic beads, for example, there are mentioned such as Streptavidin(SA) Magnetic beads: DYNABEADS MYONE (magenetic beads) Streptavidin C1 and the like. About 8 to 15 volume of the magnetic beads against the concentration of mRNA—peptide conjugate used in the binding is poured into a desired volume of Protein LoBind tube (Eppendorf). Then, the tube is stood on a magnetic stand to remove the supernatant. 1× binding buffer is added to the tube for resuspension, and tube is again stood to remove the supernatant. By this, the magnetic beads are washed and RNase free.

Then, the translation products obtained as described above (mRNA-peptide conjugate) is added, and incubated at about 20 to 30° C. for about 75 to 135 minutes by using rotator with stirring. By this, mRNA-peptide conjugate is bound to the magnetic beads.

mRNA-peptide conjugate is bound onto the magnetic beads in the tube, and then the tube is stood on the magnetic stand to remove the supernatant. The procedures that the desirable amount, for example, about 50 to 150 μL of 1× binding buffer is added to the tube to resuspend the magnetic beads, and remove the supernatant is repeated in desirable numbers.

After that, cDNA is synthesized by using the desirable solution to obtain the cDNA display, wherein cDNA is bound to the mRNA-peptide conjugate. In order to synthesis such cDNA, for example, the commercially available products such as REVERTRA ACE (reverse transcriptase) (Toyobo) and the like may be used. For example, the reaction buffer for cDNA synthesis containing REVERTRA ACE (reverse transcriptase) attached buffer, dNTP mixture, Rever Tra Ace (100 U/μL), and ultra-pure water is added to the tube containing the pre-washed magnetic beads on which mRNA-peptide conjugate is fixed, and incubated with stirring at about 40 to 45° C. for about 60 to 120 minutes by using the rotator. By this, cDNA displays, on which cDNA is further bound, may be obtained as those fixed on the magnetic beads.

The enzyme to release the cDNA display and tag for the purification of the released cDNA display are added to the tube containing the magnetic beads binding cDNA display obtained as described above is bound, and then the tube is incubated. Here, deoxy inosine, ribopyrimidine such as ribo G, ribo T, ribo C, and ribo U may be used as the enzyme at the desirable concentration, for example, 500 to 1,500 U/μL. Also, as the tag, His-tag, Y-tag, and the like may be used. Preferably, the incubation is conducted about 36 to 38° C. for 15 to 45 minutes with stirring by using the rotator.

cDNA display obtained as described above is classified by using the tag into those expressing the VHH sequence coding peptides and those not expressing them. For example, His Mag Sepharose Ni is added to the tube including the cDNA display and incubated with shaking at the desirable temperature for desirable time period, for example, about 20 to 30° C. for 0.5 to 1.5 hour. After that, the tube is stood on the magnetic stand to remove the supernatant. The beads are washed with the buffer for tag; then, the elution buffer is added and incubated with shaking at the desirable temperature for the desirable time period, for example, at room temperature for 15 to 60 minutes. After the incubation, cDNA display expressing VHH peptides are solely obtained by collecting the supernatant.

Subsequently, the solution including the cDNA display expressing VHH peptides is subjected to buffer exchange. For the buffer exchange, for example, Micro Bio-Spin™ 6 column (BIO-RAD) may be used.

Also, gel-electrophoresis is carried out in the following procedure. According to the conventional method, 10×SDS running buffer, 1.5 M Tris-HCl buffer (pH 8.8), 0.5 M Tris-HCl buffer (pH 6.8), 2×SDS sample buffer, and 40% acrylamide solution are prepared. By using them, a stacking gel and separating gel, each gel has the desirable concentration, for example, 4% of the stacking gel and 15% of th separating gel are prepared.

By using the gel, gel electrophoresis is conducted under the desirable electric current for the desirable time period, for example, at 20 mA, the time period suitable for the separation of the samples. After completion of the electrophoresis, staining of gel is conducted by using staining solution such as FITC, SYBR-gold, and the like according to the conventional method; then results from the gel-electrophoresis are analyzed by using an imager, for example, Typhoon FLA 9500 (GE Health care Japan) and the like.

After that, binding properties of the expressing VHH peptides against the desirable sugar, for example, cancer related monosaccharide, N-acetyl-D-glucosamine (GlcNAc) is confirmed as follows. Firstly, the magnetic beads and the biotinized GlcNAc are incubated to prepare the magnetic beads on which biotinized GlcNAc are fixed, and the magnetic beads on which nothing are fixed. Firstly, the biotin-DNA is added to them in the equal volume and then incubated respectively, it is confined that the biotinized GlcNAc is fixed on the magnetic beads.

The magnetic beads is washed with the glucosamine washing buffer, and incubated with biotin at the desirable temperature and for the desirable time period, for example, about 20 to 30° C. for about 15 to 45 minutes. After that, they are stirred with shaking by using the shaker, cDNA display specifically binds to GlcNAc is obtained.

Fixation of the biotinized GlcNAc onto the magnetic beads is confirmed by using the desirable carbohydrate recognition peptide, for example, a DNA fragment, PDO which is constructed by using POU domain of Oct1 protein. As shown in FIG. 5, PDO comprises the sequences such as T7, 5′ cap, Ω, Kozak, GGGS (SEQ ID NO: 25), His-Tag, GGS and Y-tag, it is the peptide having the nucleotide sequence shown in below (SEQ ID NO: 10 in the sequence listing).

DNA sequence of PDO
(SEQ ID NO: 10)
5′-GATCCCGCGAAATTAATACGACTCACTATAGGGGAAG
TATTTTTACAACAATTACCAACAACAACAACAAACAACAA
CAACATTACATTTTACATTCTACAACTACAAGCCACCATG
GATAACAAATTCAACAAAGAACAACAAAATGCTTTCTATG
AAATCTTACATTTACCTAACTTAAACGAAGAACAACGCAA
TGGTTTCATCCAAAGCCTAAAAGATGACCCAAGCCAAAGC
GCTAACCTTTTAGCAGAAGCTAAAAAGCTAAATGATGCTC
AAGCACCAAAAGCTGACAACAAATTCAACGGGGGAGGCAG
CCATCATCATCATCATCACGGCGGAAGCAGGACGGGGGGC
GGCGGGGAAA-3′

By using PDO to which biotin is bound according to the conventional method, it is decided whether GlcNAc is bound to the magnetic beads or not. The PDO concentration before they are placed in the tube containing the magnetic beads, and the DNA concentration in the supernatant after incubation are compared. Significant decrease of the PDO concentration after the incubation shows that PDO binds to the magnetic beads.

In vitro selection is repeated by using cDNA display expressing VHH peptides and the magnetic beads on which GlcNAc, cDNA display expressing VHH peptides binding to GlcNAc are obtained.

### EXAMPLES

### (Example 1) Preparation of the Linker of the Present Invention (Puromycin-Linker (Poly A+cnvK)) and Evaluation of their Properties

(1) The Preparation of the Linker of the Present Invention (Puromycin-Linker (Poly A+cnvK))

The structures of puromycin-linker (poly A+cnvK) are shown in FIGS. 3A and 3B. Biotin fragment (poly A+cnvK: the molecular backbone) has the sequence shown as the SEQ ID NO: 1 or 26 in the sequence listing. Here, BioTEG is bound to 5′ end of the molecular backbone. Also, N in the nucleotide sequence represents inosine, X represents Amino C6-dT, respectively. The puromycin-segment (a side chain) has the following sequence.

5′ (5S)TCTFZZCCP

P located at the free and of the side chain, represents puromycin as the protein binding site. Also, in the following nucleotide sequence, (5S) represents 5′ Thiol C6, F represents FITC-dT, and Z represents Spacer 18, respectively. The chemical synthesis both of the molecular backbone and the side chain were ordered to Tsukuba Oligo Service Co., Ltd.

Firstly, 15 nmol of biotin fragment (poly A+cnvK) (final conc. 150 μM) and EMCS (Dojindo Molecular Technologies, Inc, final conc. 16.7 mM) were added to 0.2 M pf sodium phosphate buffer (pH 7.2), and incubated at 37° C. for 30 minutes. After that, ethanol precipitation was conducted by using Quick-Precip Plus Solution (Edge BioSystems).

Next, 37.5 nmol of puromycin-segment was dissolved in 1 M of sodium hydrogen phosphate solution containing 50 mM DTT so as that final concentration becomes 417 M, and stirred at room temperature for 1 hour by using the shaker. Then, the solution was subjected to the buffer change to 0.1 M pf sodium phosphate (pH 7.0) containing 0.15 M of NaCl by using NAP5 column.

The reduced puromycin-segment of which buffer was already exchanged was mixed with the ethanol precipitate of the EMCS-modified biotin—fragment (poly A+cnvK), and stood at 4° C. overnight. Subsequently, DTT was added to the solution so as to become final concentration at 50 mM, and stirred at room temperature for 30 minutes. After that, ethanol precipitation was conducted by using Quick-Precip Plus Solution (Edge BioSystems). The ethanol precipitation products were dissolved in 100 L of Nuclease-free water (Nacalai Tesuque, Inc.), and subjected to HPLC purification by using C18 column.
A solution: 0.1 M of trimethyl ammonium acetate (ultra-pure water)
B solution: 80% acetonitrile
Program: Composition ratio of A solution and B solution is gradient one, wherein 85% of A solution decreases to 65% over 45 minutes
Flow rate: 1 mL/minute
Fraction: 1 mL

The components in the fractions were detected by using the fluorescence and UV absorbance (280 nm). The fractions from 30 to 32 minutes showed peaks in both of the fluorescence and UV absorbance. The fractions from 30 to 32 minutes were collected, and then solvent was evaporated by using the vacuum evaporator. Then, ethanol precipitation was conducted by using Quick-Precip Plus Solution, and the precipitate was dissolved in Nuclease-free water to be stored at −20° C.

(2) Manufacturing of Hybridize Preparation of BDA mRNA and Puromycin-Liker (Poly A+cnvK)

Transcription was conducted by using RiboMAX Large Scale RNA Production Systems-T7 (Promega Corp.). BDA mRNA obtained by the transcription and puromycin-linker (poly A+cnvK) were added to 25 mM Tris-HCl buffer (pH 7.5) containing 100 mM NaCl so as that final concentration becomes 1 μM, respectively. The buffer was incubated at 90° C. for 1 minute, then it was incubated at 70° C. for 1 minute, and the temperature was decreased to 25° C. at the rate of 0.08° C./second to hybridize the puromycin-linker (poly A+cnvK) to 3′ terminal of mRNA. Subsequently, UV (366 nm) was irradiated by using CL-1000 UV Crosslinker for about 1 minute. By these procedures, the hybridize preparation of BDA mRNA and the puromycin-linker (poly A+cnvK).

Next, the preparation (the solution) was divided into two portions to prepare the samples. For one sample, UV (366 nm) was irradiated by using CL-1000 UV Crosslinker for about 2 minutes. For the other sample, UV was not irradiated. Three pmol of each sample were taken, and incubated at 30° C., for 10 or 20 minutes by using L scale of the cell free translation system (Rabbit reticulocyte Lysate (nuclease-treated), Promega Inc.). Then, MgCl₂ and KCl were added to them at the final concentration of 75 mM and 900 mM, respectively, and incubated at 37° C. for 1 hour to form the mRNA-peptide conjugate.

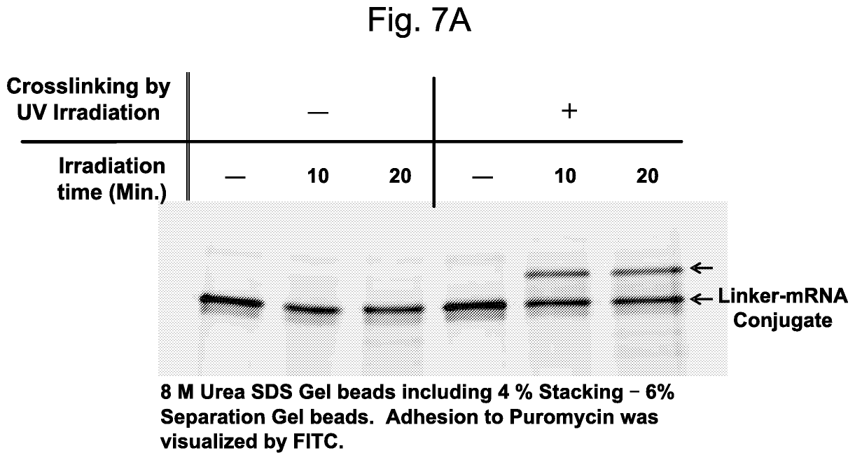

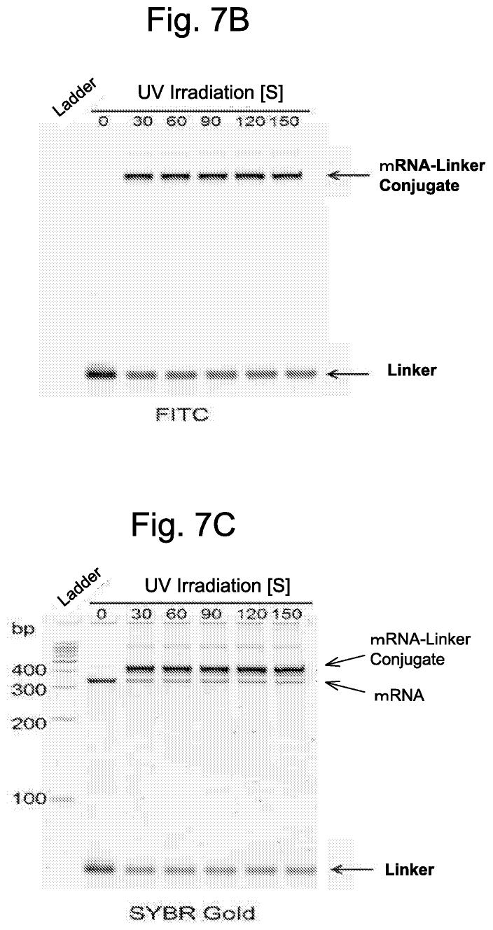

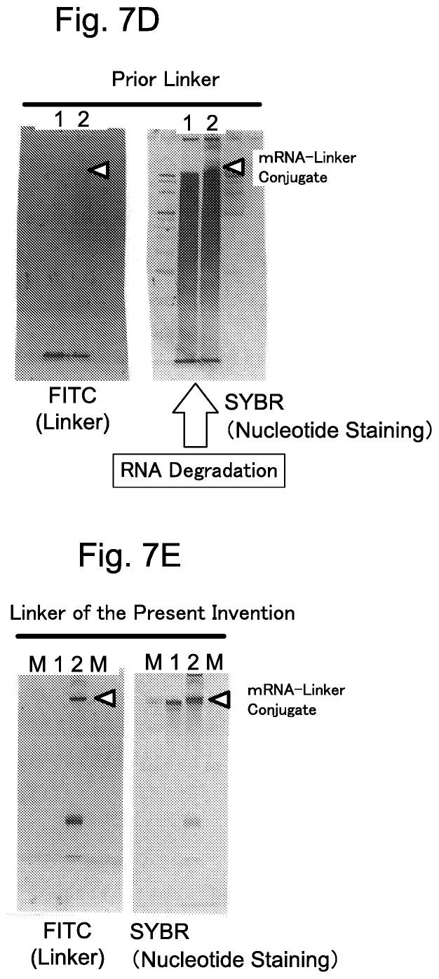

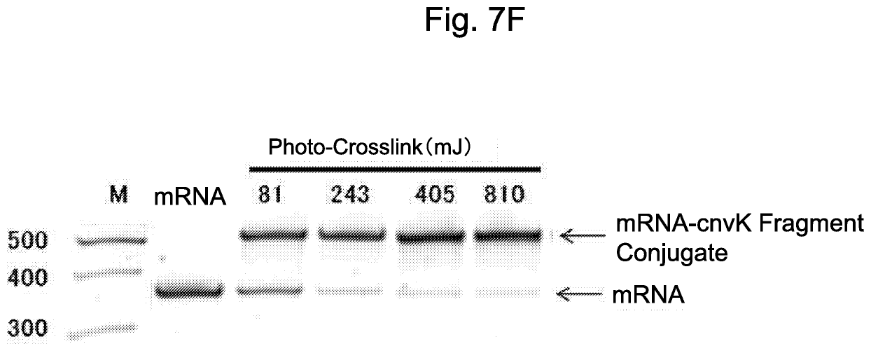

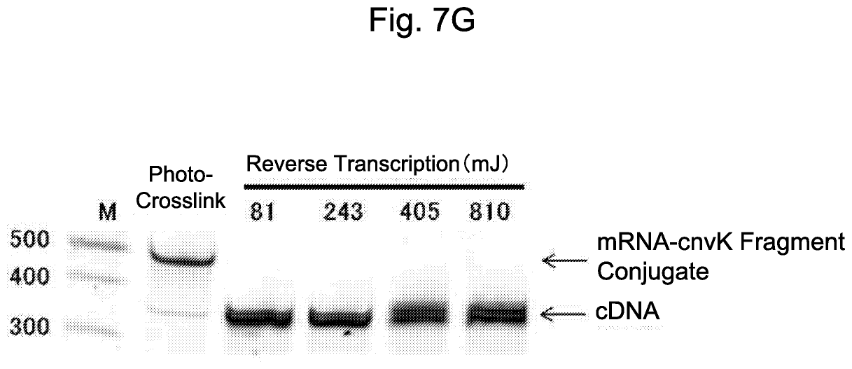

SDS-PAGE was conducted by using 4% stacking gel containing 8 M urea—6% separating gel to confirm whether the mRNA-peptide conjugate was formed or not with SYBR Gold staining (see, FIG. 7A). UV irradiation time was set to 0 minute, 10 minutes or 20 minutes. As shown in FIG. 7A, in the preparation without UV irradiation, the formation of the mRNA-peptide conjugate was not observed at any time points. In contrast, in the preparation with UV irradiation, the formation of the mRNA-peptide conjugate was observed at all of the time points.

### (Example 2) Study of Irradiation Amount in the Photo-Cross-Linking

As a model of the study, DNA coding B domain of A protein (Sequence No. 6 in sequence listing) was used. RiboMAX Large Scale RNA Production Systems-T7 was used for the transcription thereof. BDA mRNA obtained from the transcription and the puromycin-linker (poly A+cnvK) were added into 25 mM Tris-HCl buffer (pH 7.5) containing 100 mM NaCl so as that their final concentration became 1 μM, respectively. The buffer was incubated at 90° C. for 1 minute, and it was incubated at 70° C. for 1 minute, and then the temperature was decreased to 25° C. at the rate of 0.08° C./second to hybridize the puromycin-linker (poly A+cnvK) to the 3′ terminal of mRNA. After that, UV having 366 nm of wavelength was irradiated for 30 to 150 seconds by using CL-1000 UV Crosslinker to study the time necessary for the cross-linking (FIGS. 7B and 7C).

SDS-PAGE was conducted by using 4% stacking gel containing 8 M urea—6% separating gel to confirm whether the mRNA-peptide conjugate was formed or not with FITC or SYBR Gold staining (see, FIG. 7B). Both of the staining results showed that the mRNA-linker was formed.

As shown in FIG. 7B, it was confirmed that less than 30 seconds were enough to ligate mRNA and the linker as UV irradiation time thereof. In order to minimize the damage against the linker and to form stable photo-cross-linking, UV irradiation time was set to 2 minutes.

### (Example 3) Model Selection of FLAG Epitope

### (1) Transcription of the Library

DNA coding 8 amino acids random library DNA having the following sequence (SEQ ID NO: 2 in sequence listing) and DNA coding FLAG sequence (SEQ ID NO: 11 of sequence listing) were mixed at the molar ratio of 50,000:1 to prepare DNA mixture. 500 ng of the DNA mixture was taken and used for the transcription by using RiboMAX Large Scale RNA Production Systems-T7 (hereinbelow, it was sometimes simply referred to as “Kit”) in 15 μL scale. In the following sequence, optionally represents A, T, G, or C, and K represents G or T.

[SEQ ID NO: 2: 8 amino acids random library DNA]
GATCCCGCGAAATTAATACGACTCACTATAGGGGAAGTAT
TTTTACAACAATTACCAACAACAACAACAAACAACAACAA
CATTACATTTTACATTCTACAACTACAAGCCACCATGGGC
AGCNNKNNKNNKNNKNNKNNKNNKNNKGGAGGTGGAATTA
AAAACATGTGCAATTTGAACCCACTTTTAAAAAAGTGGCT
AAATGATGCAAAGGGGGGAGGCAGCCATCATCATCATCAT
CACGGCGGAAGCAGGACGGGGGGCGGCGTGGAAA
[SEQ ID NO: 11: FLAG DNA sequence]
GATCCCGCGAAATTAATACGACTCACTATAGGGGAAGTAT
TTTTACAACAATTACCAACAACAACAACAAACAACAACAA
CATTACATTTTACATTCTACAACTACAAGCCACCATGGGC
AGCGATTATAAGGACGATGACGATAAGGGGAGGTGGAATT
AAAAACATGTGCAATTTGAACCCACTTTTAAAAAAGTGGC
TAAATGATGCAAAGGGGGGAGGCAGCCATCATCATCATCA
TCACGGCGGAAGCAGGACGGGGGGCGGCGTGGAAA

The mixture described above was incubated at 37° C. for 4 hours, and then 0.5 μL of DNase(RQ1 DNase) attached to the Kit was added to it, and further incubated at 37° C. for 10 minutes. Synthesized mRNA was purified by using After Tri-Reagent RNA Clean-Up Kit.

### (2) Photo-Cross-Linking

Similar to Example 2, mRNA and puromycin-linker (poly A+cnvK) were photo-cross linked, and 20 pmol of the library mRNA was photo-crosslinked to puromycin-linker (poly A+cnvK). The irradiation time for the photo-cross-linking was set to 2 minutes.

Also, RNA decomposition was studied by using the conjugate of the cnvK linker of the present invention and mRNA or the linker (SBP) of the prior art 1 and T4 RNA ligase. Firstly, mRNA was ligated to the linker of the prior art 1 in the buffer at 25° C. for 30 minutes. The buffer was subjected to gel-electrophoresis under the conditions of 200 V, 15 mA, 8 M urea denaturing 4% acrylamide gel, and the results showed that mRNA was decomposed with SYBR staining (FIG. 7D). In contrast, when the cnvK linker of the present invention was used, the result showed that mRNA was not decomposed at all (FIG. 7E). The reason was considered that the cnvK linker made the ligation reaction thereof in water without metal ions such as zinc and the like, not but buffer with those; thereby it did not activate RNase which was activated in the buffer.

Also, UV was irradiated so as to 81 mJ to 810 mJ of the irradiation amount, damages for mRNA by photo-cross-linking were confirmed (FIG. 7F). As shown in FIG. 7F, the degradation of mRNA-cnvK linker fusion body was not observed. Furthermore, influence of UV irradiation amount for the cDNA synthesis was studied. The results showed that synthesized amount of cDNA was not changed, as the same as the damages against mRNA (FIG. 7G).

### (3) Translation by Using the Cell Free Translation System

As the same as that in Example 2, 15 pmol of the mRNA-linker conjugate was translated at 30° C. for 15 minutes in 125 μL scale by using the cell free translation system as described above. After that, MgCl₂ and KCl were added to the buffer so as that the final concentrations thereof became 75 mM and 900 mM, respectively at 37° C. for 1 hour to obtain the translation reaction buffer containing the mRNA-peptide conjugate.

### (4) Purification and Reverse Transcription

0.5 M EDTA (pH 8.0) was added to the translation reaction buffer obtained as describe above so as that the final concentration of it became 83 mM and incubated at room temperature for 5 minutes to remove ribosomes bound to the mRNA-peptide conjugate.

Subsequently, equal volume of 2× binding buffer for SA (20 mM Tris-HCl (pH 7.5) containing 2 M NaCl, 2 mM EDTA, and 0.2% Tween-20) was added to the translation reaction buffer treated as described above, and it was mixed with 150 μL of DYNABEADS MYONE (magenetic beads)C1 streptavidin (Thermo Fischer Scientific) already washed with 1× binding buffer for SA, and then it was stirred at 25° C. for 30 minutes by using the cooled thermo block rotator (Nissinrika Inc., SNP-24B).

DYNABEADS MYONE(magenetic beads) C1 streptavidin described above was washed with 200 μL of 1× binding buffer for SA three times, and then 100 μL of the reverse transcription solution was added thereto according to the protocol attached with REVERTRA ACE (reverse transcriptase) (Registered trademark). Then, the reverse transcription was conducted by using the cooled thermo block rotator at 42° C. for 15 minutes to prepare the mRNA/cDNA-peptide conjugate.

### (5) Recovery of the Reverse Transcription Products on the Magnetic Beads

DYNABEADS MYONE (magenetic beads) C1 streptavidin reacted in (4) described above was washed with 150 μL of 1×NE buffer, and then 75 μL of 1×NE buffer containing 10 U of Endonuclease V (New England Bio Labs Japan Inc.) was added. They were stirred at 37° C. for 1 hour by using the cooled thermo block rotator. Next, 75 μL of 2× His-tag washing buffer (40 mM sodium phosphate buffer (pH 7.4), containing 1 M NaCl, and 0.1% of Tween-20), and then the supernatant was recovered.

### (6) Purification by Using His-Tag

150 μL of the recovered supernatant was mixed with 20 μL of His Mag Sepharose Ni (GE Health Care, already washed with 1× His-tag washing buffer), and stirred by using Intel mixer RM-2M (Toho Inc.) at room temperature of for 1 hour. The beads were washed with 100 μL of 1× His-tag washing buffer twice, and then, EDTA-enrich L of the selection buffer (50 mM Tris-HCl buffer (pH 7.4) containing 1M NaCl, 10 mM imidazole, 5 mM EDTA and 0.1% Tween-20) was added, and then stirred by using the Intel mixer RM-2 M at room temperature for 10 minutes to recover the supernatant.

### (7) Affinity Selection

MicroSpin Empty Columns (GE Health Care) was filled with 50 μL of anti-FLAG M2 affinity gel (50% suspension), and then washed with 200 μL of the selection buffer three times. After that, 100 μL of the supernatant was loaded on the column, and stirred by using the rotator at room temperature for 1 hour. The column was washed with 200 μL of the selection buffer 4 times, and then 100 μL of 3×FLAG peptide (100 ng/μL) (Sigma-Aldrich Japan) was added, and stirred by using the rotator at room temperature for 15 minutes to competitively elute mRNA/cDNA-peptide conjugate bounds to anti-FLAG M2 affinity gel.

Elution buffer remained in the column was recovered by the centrifugation. Then, the recovered buffer was conducted to ethanol precipitation by using Quick-Precip Plus Solution, and dissolved in 15 μL of Nuclease-free water. The ethanol precipitates were added to 200 μL of the PCR reaction mixture (1× PrimeSTAR buffer (with Mg²⁺) containing 0.2 mM dNTPs, 0.4 μM T7Ω new, 4 μM NewYtag for cnvK, 0.02 U/μL of PrimeSTAR HS DNA polymerase), the following PCR program was executed. The sequences of T7Ω new and NewYtag for cnvK were shown in below (SEQ ID NO: 3 and 4 in sequence listing).

[SEQ ID NO: 3: T7Ω new sequence]
5′-GATCCCGCGAAATTAATACGACTCA
CTATAGGGGAAGTATTTTTACAACAATT
ACCAACA-3′
[SEQ ID NO: 4: NewYtag sequence for cnvK]
5′-TTTCCACGCCGCCCCCCGTCCTGCTTCCGCCGTGATGAT-3′

Next, the executed PCR program was shown: (a1) at 98° C. for 1 minute, (b1) at 98° C. for 15 seconds, (c1) at 68° C. for 30 seconds, (d1) at 68° C. for 1 minute, and both (b1) and (c1) were conducted 25 cycles to obtain the PCR products. The PCR products as described above was electrophoresed by using 8 M urea denaturing 6% PAGE, then the full construct DNA (274 mer) was excised and purified according to the conventional method. The purified full construct DNA was added to 200 μL of PCR reaction mixture (1× PrimeSTAR buffer (with Mg²⁺) containing 0.2 mM dNTPs, 0.4 μM Newleft, 0.4 μM NewYtag for cnvK, 0.02 U/μL of PrimeSTAR HS DNA polymerase), the mixture was dispensed by 50 μL portions. Then, the following PCR program was executed to obtain the double strand DNA. In below, the sequence of Newleft is shown (SEQ ID NO:. 5 in sequence listing).

[SEQ ID NO: 5: Newleft sequence]
GATCCCGCGAAATTAATACGACTCACTATAGGG

The PCR program was (a2) at 98° C. for 1 minute, (b2) at 98° C. for 10 seconds, (c2) at 68° C. for 30 seconds, (d2) at 68° C. for 1 minute, and the steps (b2) and (c2) were conducted 5 cycles.

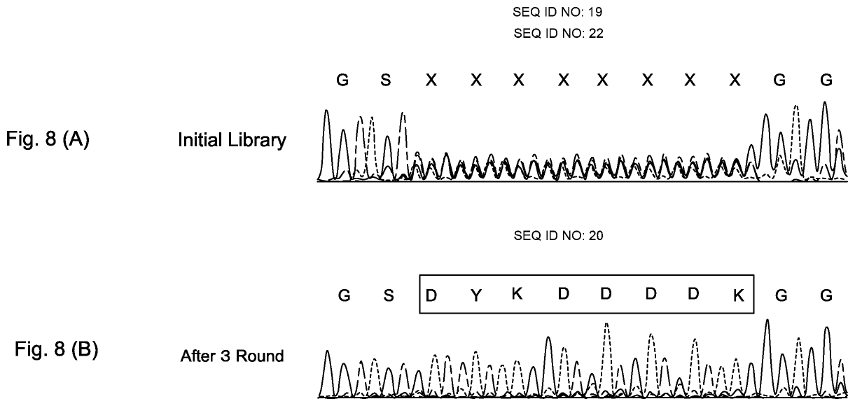

The PCR reaction mixtures in 4 tubes were pooled and purified by using the column, and used in the coming round. From the steps (1) transcription of the library DNA to (7) affinity selection were conducted 3 rounds in total, the resulting full construct DNA obtained as described above was subjected to the direct sequencing, which was ordered to Eurofins Genomics K.K. From the results of the direct sequencing, it was confirmed that FLGA DNA was converged (FIG. 8(A) and 8(B)).

### (Example 4) Manufacturing of Biotin Adhesion Protein and Apply to SPR Device

In the example, as the same as described above, B domain of A protein (BDA) was used as the model protein. By using the same procedure from (1) to (7) of the model selection of the FLAG sequence for cDNA display method in Example 3, 30 pmol worth of the mRNA-peptide conjugate was prepared by using BDA coding DN.

Next, 1/9 volume of 10×NE buffer 2 and 20 U RNase H(Takara Bio) were added to the translation products containing the mRNA-peptide conjugate, and incubated at 37° C. for 1 hour to degrade mRNA. The equal volume of 2× binding buffer for oligo dT (20 mM Tris-HCl (pH 7.5) containing 1M LiCl, 2 mM EDTA, and 0.1% Tween-20) to the mixture was mixed with 100 μL of DYNABEADS (magenetic beads) Oligo(dT)25 (SEQ ID NO: 24) (already washed with 2× binding buffer for oligo dT, Thermo Fisher Scientific) and stirred by using the rotator at room temperature for 30 minutes.

DYNABEADS (magenetic beads) Oligo(dT) 25 (SEQ ID NO: 24) was washed with 200 μL of 1× binding buffer for oligo dT twice, and then 232 μL of ultra-pure water was added and incubated at 37° C. for 10 minutes to elute the biotin adhesion protein (herein below, it is sometimes referred to as “biotin adhesion BDA”). Equal volume of 2× His-tag washing buffer to the biotin adhesion BDA solution obtained as described above was added thereto, and then mixed with 30 μL worth of His Mag Sepharose Ni (already washed with 1× His-tag washing buffer), and stirred by using the rotator at room temperature for 40 minutes.

The beads were washed with 100 μL of 1× His-tag washing buffer twice, and then, 56 μL of His-tag elution buffer (20 mM sodium phosphate buffer (pH7.4) containing 0.5 M NaCl, 250 mM imidazole, 0.05% Tween-20) was added, and stirred by using Intel mixer RM-2M at room temperature for 15 minutes to elute the biotin adhesion BDA. DNA sequence of the obtained biotin adhesion BDA was as follows (SEQ ID NO: 6 in sequence).

[SEQ ID NO: 6: BDA DNA sequence]
GATCCCGCGAAATTAATACGACTCACTATAGGGGAAGTAT
TTTTACAACAATTACCAACAACAACAACAAACAACAACAA
CATTACATTTTACATTCTACAACTACAAGCCACCATGGAT
AACAAATTCAACAAAGAACAACAAAATGCTTTCTATGAAA
TCTTACATTTACCTAACTTAAACGAAGAACAACGCAATGG
TTTCATCCAAAGCCTAAAAGATGACCCAAGCCAAAGCGCT
AACCTTTTAGCAGAAGCTAAAAAGCTAAATGATGCTCAAG
CACCAAAAGCTGACAACAAATTCAACGGGGGAGGCAGCCA
TCATCATCATCATCACGGCGGAAGCAGGACGGGGGGCGGC
GTGGAAA

### (Example 5) Affinity Assay by Using Biotin Adhesion Protein

A sensor chip SA was set to Biacore X100 (GE Health Care). The biotin adhesion BDA solution obtained as described above was diluted to 2 fold with 1× His-tag washing buffer. In order to immobilize the biotin adhesion BDA on the sensor chip, the diluted biotin adhesion BDA solution run through at the flow rate 5 μL/minute for 900 seconds.

Next, 2 fold serial dilutions such as 160 nM, 80 nM, 40 nM, 20 nM, and 10 nM of IgG obtained from rabbit serum (Sigma-Aldrich) were prepared by using 1×HBP-EP+buffer (GE Health Care). The liquids from these serial dilutions were injected into both of Fc1 and Fc2 channels at the flow rate of 5 μL/minute, and the affinities were assayed. Sensorgram of the assay channel (Fc2) was obtained by using both of the buffer control and Fc1 (control), and overlaid for kinetic fitting, and the rate at the binding (kₐ) and dissociating (k_{d}), and affinity (K_{D}=ₖ/kₐ) were obtained. The kinetic fitting was performed by using Biacore software which employed 1:1 Langmuir binding model (A+B=AB). All of the experiments were repeated 3 times and mean K_{D} with standard deviation was shown.

The sensorgram assay employing multicycle method wherein glycine—HCl (pH 2.0) was used as the regenerating solution. On the basis of the obtained sensorgram, kinetic analysis and affinity analysis were respectively conducted (FIGS. 8A and 8B). The resulting K_{D} was 1.923×10⁻⁸, and it was almost the same as that in the reference data. This means that the molecule binds to the cnvK linker of the present invention shows the inherent affinities, when the cnvK linker of the present invention is used.

### (Example 6) Synthesis of the Biotin-cnvK Linker and Preparation of the Peptide Aptamer

### (1) Synthesis of the Biotin-cnvK Linker

The cnvK linker used in the present example (see FIG. 3B) which is composed of the segment comprising cnvK (the molecular backbone) and Puro-F-S(the side chain) comprising puromycin and FITC was used to prepare Biotin-cnvK linker by ligating the biotin by using the following method.

9.4 mg of EMCS (the crosslinking agent, Wako Pure Chemical Industries, Ltd.) was dissolved in 305.5 μL of N,N-dimethylformamide to prepare 100 mM EMCS. Next, 10 nmol Biotin-cnvK segment and 40 μL of 100 mM EMCS were added to 200 μL of 0.2 M sodium phosphate buffer (Molecular biology grade, pH 7.2, Wako Pure Chemical Industries, Ltd.), and then incubated at 37° C. for 30 minutes. After that, it was subjected to ethanol precipitation to concentrate the products.

Both of 80 μL of 1M Na₂ PO₄ (pH 9.0) and 10 μL 1 M DTT were added to 2 mM Puro-F-S, and stirred with shaking for 1 hour at room temperature. Then, it was subjected to the buffer exchange by using NAP 5 column filled with 20 mM phosphate buffer to collect the fractions with fluorescence solely. EMCS solution after the ethanol precipitation was added to the collected Puro-F-S, and stood 4° C. for overnight.

1/20 volume of the entire volume of the sample solution of 1 M DTT was added, and stirred with shaking at room temperature for 30 minutes. Then the solution was subjected to ethanol precipitation to concentrate the products. After the concentration, it was diluted in a measuring cylinder to 100 μL by using ultra-pure water, and subjected to HPLC under the same conditions as those in Example 1 to purify the biotin-cnvK linker.

### (2) Preparation of the Crosslinked Peptide Aptamer

### (2-1) DNA Amplification by PCR Employing VHH Library as the Template

PCR 1 was conducted for CDR1, CDR2, and CDR3 under the following conditions by using VHH library having random sequence (SEQ ID NO: 7 in sequence listing) as the template DNA to amplify the DNAs. In the sequence 7, n, b, d, r, y, h, y, and m represent the same as those described before.

sequence of VHH library construct]
SEQ ID NO: 7
5′-GATCCCGCGAAATTAATACGACTCACTATAGGGGAAG
TATTTTTACAACAATTACCAACAACAACAACAAACAACAA
CAACATTACATTTTACATTCTACAACTACAAGCCACCATG
GGCGAGGTGCAGCTGGTGGAGAGCGGAGGAGGATCCGTGC
AGGCTGGAGGAAGCCTGCGCCTGAGCTGCGCTGCTAGCGG
AnbnnbndrbyhmyhmyhmdrbnbnnbnTGGTTCCGCCAG
GCTCCTGGAAAGGAGCGCGAGGGAGTGnbnnbndrbyhmy
hmyhmyhmdrbACCTACTACGCTGACAGCGTGAAGGGACG
CTTCACCATCAGCCAGGACAACGCCAAGAACACCGTGTAC
CTGCAGATGAACAGCCTGAAGCCTGAGGACACCGCTATCT
ACTACTGCGCTGCTdrbdrbyhmyhmyhmyhmyhmyhmyh
myhmyhmyhmdrbTACTGGGGACAGGGAACCCAGGTGACC
GTGGGAGGAGGCAGCCATCATCATCATCATCACGGCGGAA
GCAGGACGGGGGGCGGCGGGGAAA-3′

TABLE 3

| Composition of the solution for PCR 1 | volume (μL) |
|---|---|
| 5 x Prime STAR Buffer (Mg²⁺ plus) | 5 |
| dNTP mix (2.5 mM each) | 2 |
| 20 μM Primer 1 (New Y tag for Poly A & cnvK | 0.5 |
| Lin.) | |
| 20 μM Primer 2 (New left) | 0.5 |
| template DNA | 2 |
| Prime STAR HS DNA polymerase (2.5 U/μL) | 0.3 |
| (Takara) | |
| Ultra-pure water | 14.7 |
| Total | 25 |

PCR program contained the steps at 98° C. for 2 minutes, 5 cycles of (at 98° C. for 10 seconds, at 98° C. for 1 second, at 68° C. for 40 seconds), and at 72° C. for 2 minutes. The diversity of the original VHH was 2.7×10¹¹ molecules/μL.

PCR product 1 obtained from PCR 1 was purified by using PCR Clean-Up MiniKit (Favorgen) and remove DNA having short chain length. Next, PCR solution 2, which had the same components as PCR solution 1 except that 20 μM Primer 2 (T7 omega new) was used instead of 20 μM Primer 2 (New left), was prepared, and the purified PCR product 1 was subjected to PCR2 to obtain PCR product 2. PCR program was composed of the steps at 98° C. for 2 minutes, 23 cycles of (at 98° C. for 10 seconds, at 98° C. for 1 second, and at 68° C. for 40 seconds), and at 72° C. for 2 minutes.

(2-2) Transcription to mRNA

DNA obtained in PCR 2 was transcribed to mRNA by using RiboMAX™ Large Scale RNA production Systems (Promega). The composition of the transcription solution used in the transcription reaction and the procedure were shown in Table 4.

TABLE 4

| Composition of the solution for Transcription | Volume (μL) |
|---|---|
| T7 Transcription 5 x Buffer | 2 |
| rNTPs (25 mM ATP, CTP, GTP, UTP) | 3 |
| Template DNA | X |
| Plus Nuclease-free water | 4 - X |
| Enzyme Mix (PROMEGA) | 1 |
| Total | 10 |

The transcription solution was incubated at 37° C. for 3 hours, and 1 μL of RQ1 Rnase-Free DNAse (Promega) was added, and further incubated at 37° C. for 15 minutes. Then, mRNA was purified by using After Tri-Reagent RNA Clean-up Kit (Favorgen) according the protocol attached thereto. The procedures described above were repeated by 6^{th} round, and mRNA was obtained in each round. Here, the template DNA amount used in each round is shown in Table 5, and the amount of x in the table was varied depending on the DNA amount used. At that time, x was adjusted so as to obtain enough amount of mRNA to be used in photo-cross-linking conducted later.

TABLE 5

| Selection round | Template DNA[ng] |
|---|---|
| 1^{st} | 171 |
| 2^{nd} | 113 |
| 3^{rd} | 100 |
| 4^{th} | 26 |
| 5^{th} | 39 |
| 6^{th} | 34 |

(2-3) Binding of mRNA and Biotin-cnvK Linker

mRNA obtained in each selection round as described above was purified to obtain purified mRNA. mRNA obtained in each selection round and Biotin-cnvK linker were photo-crosslinked under the following conditions. The composition of the reaction mixture for the photo-cross-linking is shown in the following Table 6. The ratio of mRNA and the Biotin-cnvK linker in the reaction mixture of the photo-cross-linking was set to 1:1 (molar ration) for all of the cases. Annealing was conducted the process comprising the steps of at 90° C. for 2 minutes, decreasing the temperature to 70° C. for 1 minute, at 70° C. for 1 minute, and then the temperature was decreased to 25° C. for 15 minutes.

TABLE 6

| Composition of the reaction mixture | Amount used | |
|---|---|---|
| mRNA | 20 | pmol |
| Biotin-cnvK linker | 20 | pmol |
| 1M NaCl | 4 | μL |
| 0.25M Tris-HCl | 4 | μL |
| Total | 20 | μL |

After annealing, the photo-cross-linking was conducted under the condition for irradiating UV having 365 nm of wavelength at 405 mJ/cm² with CL-1000 Ultraviolet Crosslinker to obtain the conjugate of mRNA obtained in each round and the Biotin-cnvK linker (mRNA-linker conjugate).

(2-4) Formation of mRNA-Peptide Conjugate in the Cell Free System

The mRNA-linker conjugate obtained as described above was translated in the cell free translation system, and the mRNA-peptide conjugate was formed. The composition of the reaction solution used for the cell free system is shown in the following Table 7.

TABLE 7

| Composition of translation solution | Amount used | |
|---|---|---|
| Translation Mix | 0.5 | μL |
| mRNA-linker conjugate | 3 | pmol |
| Retic Lysate 1 | 17.5 | μL |
| RNase inhibitor (Promega) | 0.5 | μL |
| Ultra-pure water | added so as that final volume becomes | |
| | 25 | μL |
| Total | 25 | μL |

The tube containing the translation solution as described above was incubated at 30° C. for 20 minutes, and then, 12 μL of 3M KCl and 3 μL of 1M MgCl₂ were added and further incubated at 37° C. for 60 minutes. Then, 10 μL of ethylene diamine tetra acetic acid (pH 8.0) was added and incubated for 37° C. for 10 minutes. Next, 50 μL of the 2× binding buffer was added to obtain the mRNA-peptide conjugate wherein the peptide synthesized in the cell free system was bound to the mRNA conjugate.

### (2-5) Immobilization on the Streptavidin Magnetic Beads

Streptavidin(SA) Magnetic beads: DYNABEADS MYONE (magenetic beads) Streptavidin Cl(magnetic beads) were washed so as to be RNase-Free. The magnetic beads were added into 1.5 mL volume of Protein LoBind tube (Eppendorf) in the amount of 10 volumes against the concentration of mRNA-peptide conjugate to be added it. Then, the tube was stood on the magnetic stand to discard the supernatant. The beads were re-suspended in the 1× binding solution, and stood on the magnetic stand. The supernatant was discarded and the magnetic beads were washed. In the tube, the translation products (mRNA-peptide conjugates) obtained in (2-4) as described above were added. The tube was incubated with stirring at 25° C. for 90 to 120 minutes by using the rotator to make the mRNA-peptide conjugate with the magnetic beads bind. The amount of the mRNA-peptide conjugate used and the incubation time were shown in the following Table 8.

TABLE 8

| Selection round | Incubate period (minutes) | Amount of mRNA-conjugate |
|---|---|---|
| 1^{st} | 90 | 53.5 |
| 2^{nd} | 120 | 35.5 |
| 3^{rd} | 120 | 17.5 |
| 4^{th} | 120 | 17.5 |
| 5^{th} | 120 | 17.5 |
| 6^{th} | 120 | 11.5 |

(2-6) Reverse Transcription to cDNA and Cleavage from the Magnetic Beads

As described above, the mRNA-peptide conjugates were immobilized on the magnetic beads. The tube containing the magnetic beads was stood on the magnetic stand to discard the supernatant. In the tube, 100 μL of the 1× binding buffer was added to re-suspend the magnetic beads, and discard the supernatant, namely the magnetic beads were washed. The washing procedure was repeated twice.

After that, 5× buffer attached to REVERTRA ACE (reverse transcriptase) (Toyobo) was diluted to 1× buffer, and 100 μL portions thereof was taken out and the same procedures were conducted to further wash the beads. Next, the reaction mixture for cDNA synthesis having the composition shown in the following Table 9 was added, and incubated with stirring at 42° C. for 90 minutes by using the rotator to obtain cDNA display which further has cDNA.

TABLE 9

| Composition of the reaction mixture for cDNA synthesis | Amount used |
|---|---|
| 5 x REVERTRA ACE (reverse transcriptase) attached | 20 μL |
| buffer | |
| 10 mM dNTP mixture | 10 μL |
| Rever Tra Ace (100 U/μL) | 2 μL |
| Ultra-pure water | 68 μL |
| Total | 100 μL |

Both of (x−1) L of 1× His-tag washing buffer and 1 μL of RNase T1 (1,000 U/μL) were added to the tube including the magnetic beads to which cDNA displays obtained above, and incubated with stirring at 37° C. for 30 minutes by using the rotator to release cDNA displays from the magnetic beads.

### (2-7) His-Tag Purification and the Recovery of the Products

cDNA displays were prepared as described above, and then they were classified into two groups: one group expressed the peptides coding VHH, and the other did not express that by using His-tag purification. Firstly, His Mag Sepharose Ni was added to the tube containing the cDNA displays and incubated with stirring by using the shaker at 25° C. for 1 hour. After that, the tube was stood on the magnetic stand to remove the supernatant. Then, the beads were washed with 100 μL of 1× His tag buffer once.

Next, 50 μL of the His tag elution buffer (including high concentration of imidazole) was added to the tube, it was incubated with shaking by using the shaker at room temperature for 30 minutes. After that, the tube was stood on the magnetic stand to recover the supernatant to collect the cDNA display expressing VHH peptide solely.

After the recovery of the cDNA displays expressing VHH peptide by using His-tag purification, the recovered cDNA displays were subjected to the buffer exchange by using Micro Bio-Spin™ 6 column (BIO-RAD) for use of in vitro selection.

Firstly, the column was centrifuged keeping the lid open at 1,000×g for 2 minutes. Then, 500 μL of binding buffer for glucosamine was added and was centrifuged at 1,000×g for 1 minute. The procedure to add the binding buffer for glucosamine was repeated 4 times, and centrifugation of the last time was conducted for 3 minutes. The cDNA displays obtained as described above were added to the column and centrifuged at 1,000×g for 4 minutes. Thus, the cDNA displays of which buffer were exchanged was recovered.

### (3) Immobilization of the Cancer-Associated Monosaccharide N-Acetyl-D-Glucosamine (GlcNAc)

The magnetic beads on which the biotinized GlcNAc was immobilized by incubating the magnetic beads and the biotinized GlcNAc; and the magnetic beads on which nothing is immobilized were prepared. The equal volume of the biotin-DNA was respectively added to the tube containing the beads for confirming whether the biotinized GlcNAc was immobilized on the magnetic beads or not.

When the selection was conducted, cDNA displays which non-specifically bounds to biotin were eliminated. The magnetic beads were washed with the washing buffer for glucosamine, and incubated together with biotin at 25° C. for 30 minutes in the tube. After that, the magnetic beads were stirred by shaking to obtain cDNA displays which specifically bounds to GlcNAc. In the following Table 10, the relationship between the cDNA amount in each round and the incubation time by using the shaker.

TABLE 10

| | | Amount of cDNA display |
|---|---|---|
| Selection Round | Incubation time (min) | (pmol) |
| 1^{st} | — | — |
| 2^{nd} | 60 | 35 |
| 3^{rd} | 60 | 17 |
| 4^{th} | 60 | 17 |
| 5^{th} | 30 | 16.5 |
| 6^{th} | 30 | 10 |

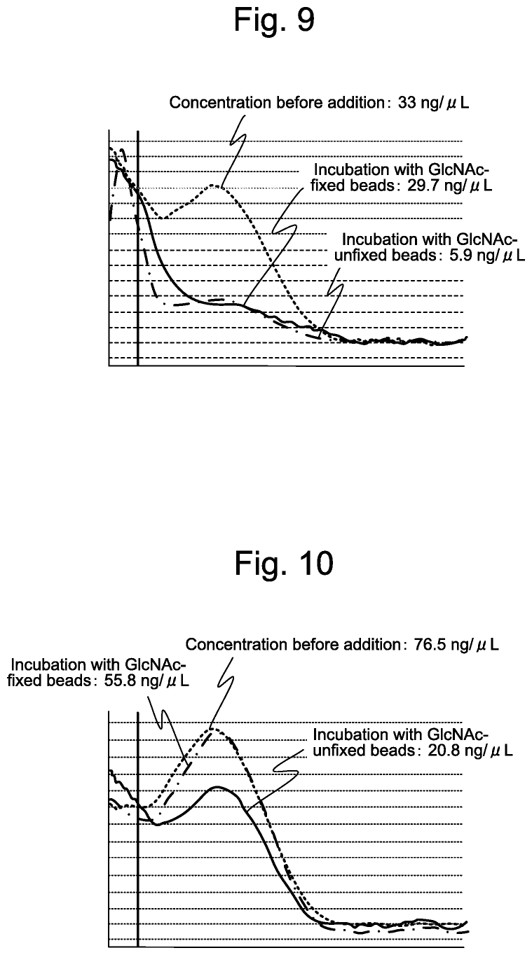

As shown in FIG. 9 and FIG. 10, the case reacted with the beads on which the biotinized GlcNAc was immobilized and the case reacted with the beads on which nothing was immobilized were compared. DNA amount in the supernatant was significantly decreased in the case reacted with the beads on which the biotinized GlcNAc was immobilized showed significant decrease of the DNA amount in the supernatant. Therefore, they showed that the biotinized GlcNAc was immobilized on the magnetic beads.

### (Example 7) In Vitro Selection

(1) Study about the Conditions for In Vitro Selection

Next, in vitro selection of VHH, of which the target molecule was GlcNAc, was conducted by using the obtained GlcNAc-specific cDNA displays in vitro. Firstly, the magnetic beads was placed in the tube and washed with the glucosamine washing buffer. Then, the biotinized GlcNAc was added to the tube and incubated with shaking at room temperature for 30 minutes. Then, the tube was stood on the magnetic stand to discard the supernatant, and the washing buffer for glucosamine was added and again the magnetic beads were washed. Next, biotin was added to the tube and incubated with stirring by using the shaker at room temperature for 30 minutes.

After termination of the incubation, the tube was stood on the magnetic stand to discard the supernatant, and the magnetic beads were washed by using the washing buffer for glucosamine. Then, the magnetic beads were incubated with stirring together with the cDNA displays at 25° C. for 30 minutes by using the rotator. Then, the tube was stood on the magnetic stand to discard the supernatant. By these procedures, cDNA displays of VHH which is GlcNAc-specific are immobilized via GlcNAc on the magnetic beads. Conditions in each round in vitro selection are shown in the following Table 11.

TABLE 11

| | Ligation Product | Immobilized amount of target |
|---|---|---|
| Selection Round | (pmol) | (pmol) |
| 1^{st} | 108 | 25,000 |
| 2^{nd} | 36 | 1,000 |
| 3^{rd} | 18 | 500 |
| 4^{th} | 9 | 100 |
| 5^{th} | 8 | 50 |
| 6^{th} | 2.8 | 50 |

As describe above, cDNA displays were immobilized on the magnetic beads via GlcNAc in the tube. Next, the washing buffer for glucosamine was added to the tube for resuspending the beads by tapping for 20 seconds. Then, the tube was stood on the magnetic stand for 1 minute to collect the supernatant. By repeating the procedures from 3 to 5 times, the collected supernatant was separately stores as the washing solution (hereinbelow, it is sometimes referred to as “Wash”.). Wash was subjected to the gel-electrophoresis to confirm the contained components. As a result, the washing times of cDNA display at each round were set as shown in Table 12. As a results of the gel-electrophoresis, washing of cDNA displays obtained after the 4^{th} round were not enough. Therefore, the washing times after the 4^{th} round were increased to remove cDNA which was not specifically absorbed.

TABLE 12

| Selection Round | Washing times |
|---|---|
| 1^{st} | 5 |
| 2^{nd} | 3 |
| 3^{rd} | 3 |
| 4^{th} | 5 |
| 5^{th} | 5 |
| 6^{th} | 5 |

Namely, enough concentration of GlcNAc against the cDNA displays immobilized on the magnetic beads were added, the elution was conducted at 4° C. for 3 days, stirring the rotator, except the 6^{th} round. At the 6^{th} round, the elution was conducted for overnight. cDNA displays of VHH which non-specifically binds to GlcNAc were removed by the washing, and then competitive elution was conducted to recover the cDNA displays of VHH which specifically binds to GlcNAc as the target.

Also, the gel-electrophoresis was conducted by using the following procedures. 10×SDS running buffer, 1.5 M Tris-HCl buffer (pH 8.8), 0.5 M Tris-HCl buffer (pH 6.8), 2×SDS sample buffer, and 40% acrylamide solution were prepared as follows.

10×SDS running buffer containing 30.3 g of Tris(hydroxymethyl)-aminomethane, 144 g of glycine, 10 g of SDS were diluted by using ultra-pure water to 1,000 mL. Also, 36.3 g of Tris(hydroxymethyl)aminomethane was dissolved in ultra-pure water and pH thereof was adjusted to 8.8 by using HCl, and then, the solution was diluted to 200 mL by using ultra-pure water to prepare 1.5 M Tris-HCl buffer (pH 8.8). 12.1 g of Tris(hydroxymethyl)aminomethane was dissolved in ultra-pure water, and pH thereof was adjusted to 6.8 by using HCl, and then it was diluted to 200 mL to prepare 0.5 M Tris-HCl buffer (pH 6.8).

12.5 mL of 0.5 M Tris-HCl buffer (pH 6.8), 5 mL of 2-mercaptoethanol, 2 g of SDS, 24 g of urea, 3 g of sucrose were dissolved in the small amount of ultra-pure water, and proper amount of bromophenol blue (herein below, it is sometimes referred to as “BPB”) was added to the solution, it was diluted to 50 mL by using ultra-pure water to obtain 2×SDS sample buffer. 194.8 g of acrylamide gel of SDS-PAGE grade and 5.2 g of bis-acrylamide were dissolved in the proper amount of ultra-pure water, which was used for dilution to 500 mL to obtain 40% acrylamide solution.

4% stacking gel (for 1 sheet) was prepared by mixing 1.25 mL of 0.5 M Tris-HCl buffer (pH 6.8), 0.5 mL of 40% acrylamide solution, and 50 μL of 10% SDS, and then it was diluted by using ultra-pure water to 5 mL. Here, 12.5 μL of 20% APS (Ammonium peroxodisulfate: ammonium peroxodisulfate) and 6 μL of TEMED (N,N,N,N-Tetramethy-ethylenediamine: N,N,N,N-Tetramethy-ethylenediamine) was added.

15% separating gel (for 1 sheet) was prepared by mixing 2.5 mL of 1.5 M Tris-HCl buffer (pH 8.8), 3.75 mL of 40% acrylamide solution, and 100 μL of 10% SDS, and then it was dilutes to 10 mL by using ultra-pure water. Then, 25 μL of 20% APS and 5 μL of TEMED were added.

10×SDS running buffer was diluted 10 fold to fill the gel-electrophoresis tank, and gel-electrophoresis was conducted by setting the gel in the tank at 20 mA for suitable time period for separating the samples. Then, FITC or SYBR-gold was used for staining the gel according to the conventional method by using the imager (Typhoon FLA 9500, GE Health Care Japan) to analyze the result of the gel-electrophoresis.

(2) Competitive Assay with GlcNAc Non-Specific Binding cDNA Display Molecule

GlcNAc was used as the target molecule, both of B DOMAIN OF A PROTEIN (it is sometimes referred to as “BDA”.) which does not bind to GlcNAc, and cDNA displays of VHH recovered after 6^{th} round were subjected to in vitro selection to confirm their binding abilities to GlcNAc.

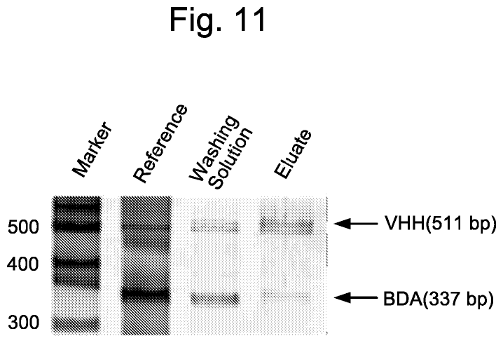

Both of the cDNA displays of VHH and the cDNA displays of BDA were added at the molar ratio of 1:1 into the tube containing the magnetic beads on which GlcNAc was immobilized, and incubated with stirring at room temperature for 30 minutes by using the rotator. Then, the tube was stood on the magnetic stand to discard the supernatant. The magnetic beads were resuspended by adding the washing buffer for glucosamine into the tube and tapping. The tube was stood on the magnetic stand for 1 minute to recover the supernatant. Next, the competitive elution was conducted by using GlcNAc to obtain the eluate, which were subjected to gel-electrophoresis with markers by using 4% PAGE, 200 V for 30 minutes, and then stained by using SYBR-gold. The result is shown in FIG. 11.

In the control before the incubation, band intensity ratio of the VHH: BDA was 9:16, and the cDNA display concentration of BDA was higher than that of VHH. After that, the washed solution showed the band intensity ratio, 1:3, and BDA concentration was high. In contrast, the eluate showed reversed band intensity ratio, 4:1, and VHH concentration became higher. The VHH concentration in the control was 0.6 of that of BDA, however, VHH concentration in the eluate showed 6 times higher compared to that of BDA.

This shows that the cDNA displays of BDA were not eluted, although the obtained cDNA display of VHH were eluted. From the results, it was confirmed that the cDNA displays of VHH obtained in vitro selection of the 6^{th} round has the binding abilities to GlcNAc.

The cross-linked peptide aptamer (both of the of cDNA displays of BDA and VHH) were prepared as the same as those in Example 6(2). After preparation of those cDNA displays respectively, both of them were added into the tube containing the magnetic beads and incubated. Then, in vitro selection was conducted as the same as the case of cDNA displays of VHH were solely added. Washing times in each round were 5, and the competitive elution was conducted at 4° C. for overnight by using GlcNAc.

### (3) Preparation of the Cross-Linked Peptide Aptamer

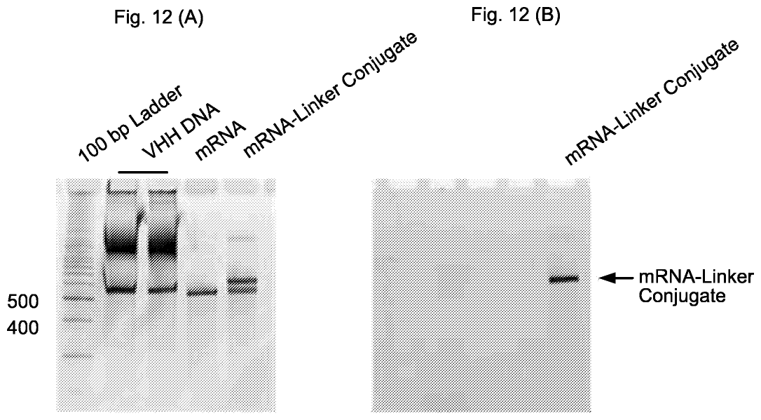

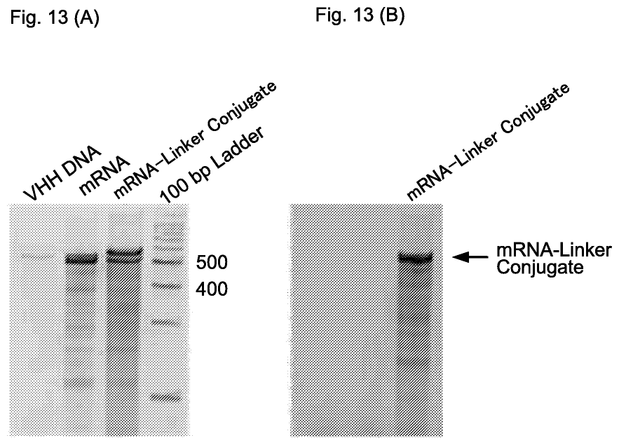

Gel electrophoresis was conducted by using PCR products of VHH library constructs obtained in the same procedure as those of Example 6(2), mRNA thereof, mRNA-linker conjugate thereof under the conditions of 4% PAGE, and 200 V for 30 minutes. The, gel was stained either of SYBR-gold or FITC to confirm the amplification by PCR, and the photo-cross-linking of mRNA and the linker. The results from the 1^{st} and 6^{th} rounds were shown as FIGS. 12(A) and 12(B),respectively, as well as FIGS. 13(A) and 13(B).

The lengths of the obtained DNAs of VHH were 541 bp, which were the same in the range of the 1^{st} round to 6^{th} round, and those of the mRNAs were also 541 mer. As shown in FIG. 13(B), it was confirmed that the sufficient amount of mRNA-linker conjugates were formed.

(4) Confirmation of mRNA-Peptide Conjugate

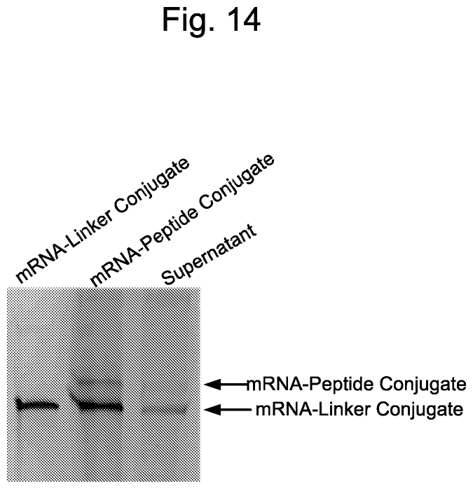

The mRNA-linker conjugate, mRNA-peptide conjugate, and magnetic beads were incubated in the test tube. After termination of the incubation, the supernatant in the tube was subjected to gel electrophoresis under the condition of 4% stacking gel, 6% running gel, 200 V for 120 minutes, and then the gel was stained by using FITC to confirm the mRNA-peptide conjugate. The mRNA-linker conjugate was used as the control. Results were shown in FIG. 14.

Compared to the band with that of mRNA-linker conjugate, the band of mRNA-peptide conjugate was shifted to upside. Therefore, the formation of the mRNA-peptide conjugate was confirmed. Also, the supernatant obtained from either of the mRNA-peptide conjugate or the magnetic beads showed faintly stained bands, which was detected upper side of those of the mRNA-linker conjugate. Therefore, the thickness of the bands were compared with that of mRNA-peptide conjugate before the incubation, of which band was detected as the thick one in the center of the gel by using Quantity One, and the ratio of the thickness of the stained bands were 7:3. As a result, on the basis of the amount of mRNA-peptide conjugate detected in the supernatant, about 57% of the mRNA-conjugate amount used was immobilized on the magnetic beads.

### (5) Confirmation of In Vitro Selection

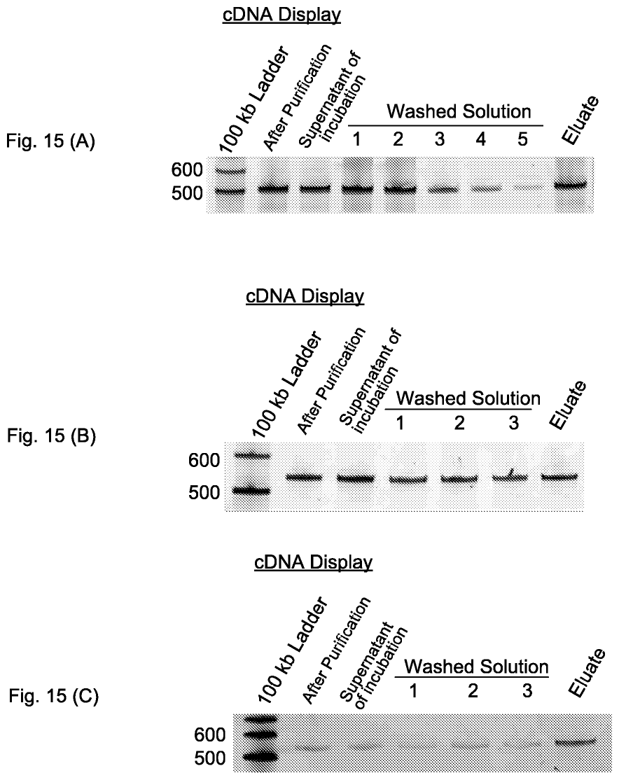

VHH which was competitively eluted by using GlcNAc in each round and the peptides contained in the washed solution were amplified by using PCR under the same conditions as those described above; and then they were subjected to gel-electrophoresis under the condition of 4% PAGE, 200 V for 30 minutes to confirm the progress of in vitro selection. The results were shown in FIGS. 15 (A) to 15 (C).

In the figures, the term, “after purification” means the purified cDNA displays. Also, the term, the “supernatant of the incubation” means the supernatant after the incubation of the magnetic beads in each round (the 1^{st} round is the column) and the cDNA displays. The term, the “washing solution” means the supernatant recovered after washing of them by tapping. Further, the term, “eluates” means the liquid eluted after the competitive elution by using GlcNAc.

The results from the 1^{st} to 3^{rd} rounds were shown in FIGS. 15 (A) to 15 (C). In the 1^{st} round, 108 pmol of the mRNA-linker conjugate were used, and the immobilized amount of the biotinized GlcNAc was 25 nmol. In the 2^{nd} round, 36 pmol of the mRNA-linker conjugate was used, and the immobilized amount of the biotinized GlcNAc was 1 nmol. In the 3^{rd} round, 18 pmol of the mRNA-linker conjugate was used, and the immobilized amount of the biotinized GlcNAc was 500 pmol. Samples from those were subjected to gel-electrophoresis under the conditions of 4% PAGE, 200 V for 30 minutes, and then gel was stained by using SYBR-gold.

It was assumed that in vitro selection progressed, because thickness of the band color on the lane of the washing solution became lighter depending on the increase of the round number, and that of the lane of the eluates did not show drastic decrease. Therefore, the incubated sample of GlcNAc non-immobilized magnetic beads and the cDNA display obtained as described above was used as the negative control after the 4^{th} round. The products from the 4^{th} to 6^{th} round were subjected to gel-electrophoresis under the condition of 4% PAGE, 200 V for 30 minutes, and then the gel was stained by using SYBR-gold.

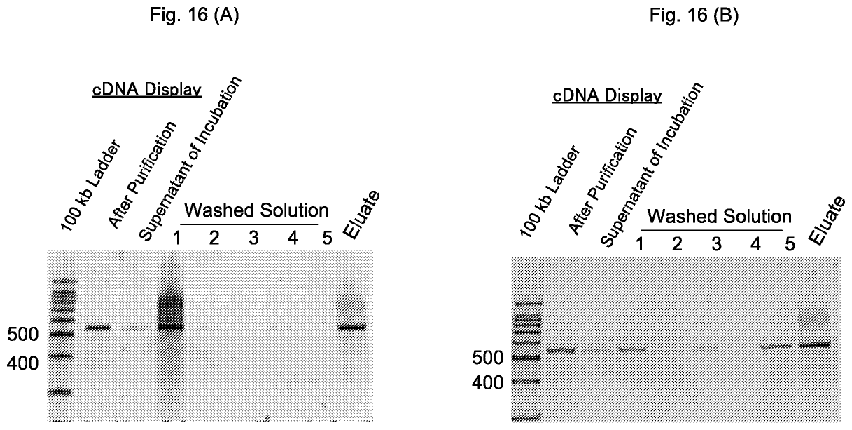

At 4^{th} round, 9 pmol of the mRNA-linker conjugate was used, and the immobilized amount of the biotinized GlcNAc was 100 nmol. Also, the incubated sample of the GlcNAc immobilized magnetic beads and the cDNA display was used as the positive control. The results were shown in FIGS. 16 (A) and 16 (B). It was judged that in vitro selection was sufficient, because the bands were observed in the lanes, wherein both of the eluates from the negative control and the positive control were loaded.

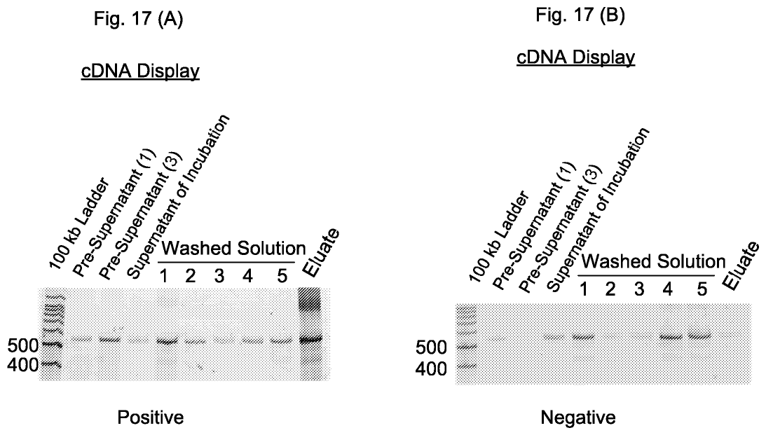

The results from the 5^{th} round were shown in FIGS. 17 (A) and 17 (B). In the 5th round, 8 pmol of the mRNA-linker conjugate was used, and the immobilized amount of the biotinized GlcNAc was 50 nmol. In this round, it was observed that the band thickness of the eluate became lighter than that of the negative control. Also, the thick band was observed in the lane to which the eluted of the positive control was loaded, and it showed the progress of the selection of GlcNAc binding to VHH.

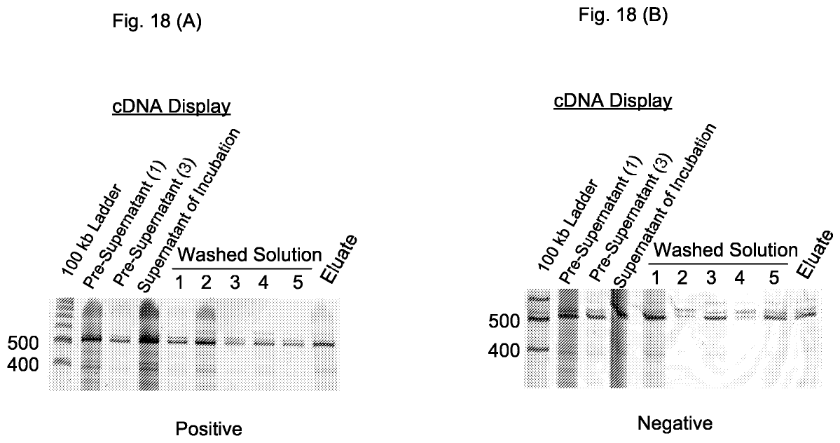

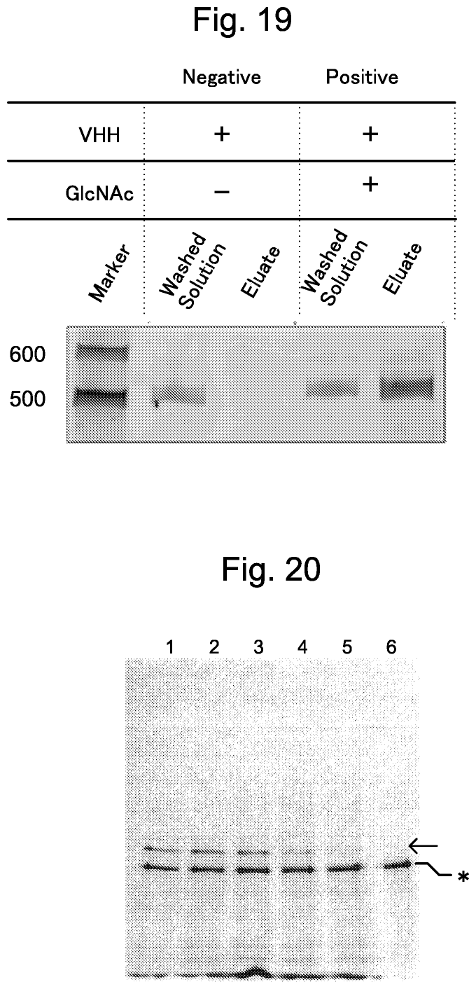

The results from the 6^{th} round were shown in FIGS. 18 (A) and 18 (B). In the 6th round, 2.8 pmol of the mRNA-linker conjugate was used, and the immobilized amount of the biotinized GlcNAc was 50 nmol. The thicknesses of the bands of the lanes, to which the washed solution of the negative control or that of the positive control was loaded, were almost the same. Therefore, the respective washed solution 5 of the negative or positive, and the eluate from them were subjected to gel-electrophoresis under the condition of 4% PAGE, 200 V for 30 minutes, and the gel was stained by using SYBR-gold. The result was shown in FIG. 19.

Also, the ratios among the band thicknesses was obtained by using the calculation software of Quantity One 1^{st} dimension gel-electrophoresis analysis software (Bio-Rad Laboratories), that of the marker 600 as B1, that of the negative as U1, and that of the positive as U2. The results were shown in the following Table 13.

TABLE 13

| | | Volume | Adj. Vol. | | |
|---|---|---|---|---|---|
| Indexes | Name | CNT*mm2 | CNT*mm2 | %Adj. Vol. | Conc. |
| 1 | U1 | 16954.74923 | 3611.166564 | 4.28 | N/A |
| 2 | U2 | 89289.14294 | 80740.91029 | 95.72 | N/A |
| 3 | B1 | 15115.77724 | 0.000000000 | N/A | N/A |

As shown in Table 13, the eluate of the positive was 22 times thicker than that of the negative. As a result, it was confirmed that the eluate of the positive contains GlcNAc binding VHH. Also, from the result of the sequence analysis, it was indicated that CDR3 contributes to the recognition of GlcNAc.

### (Comparative Example) Preparation of the Linkers of the Prior Arts

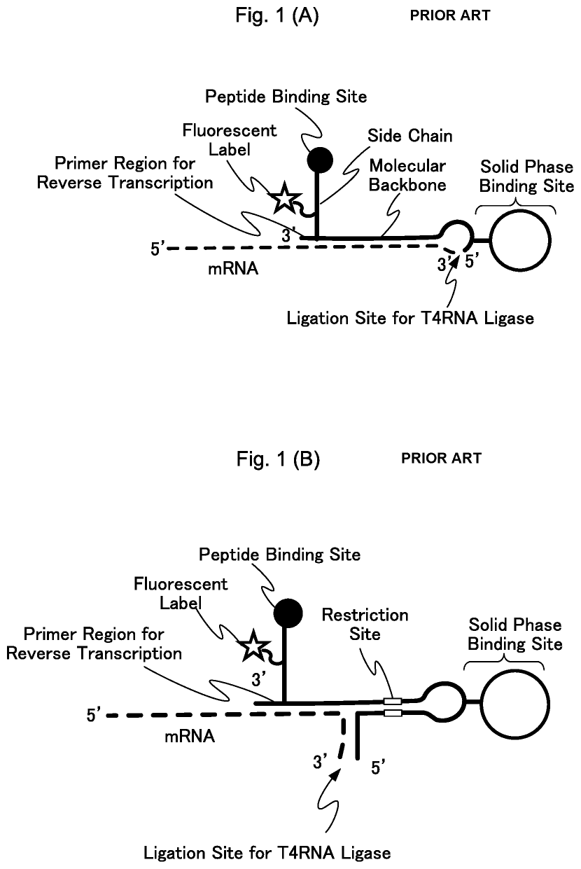

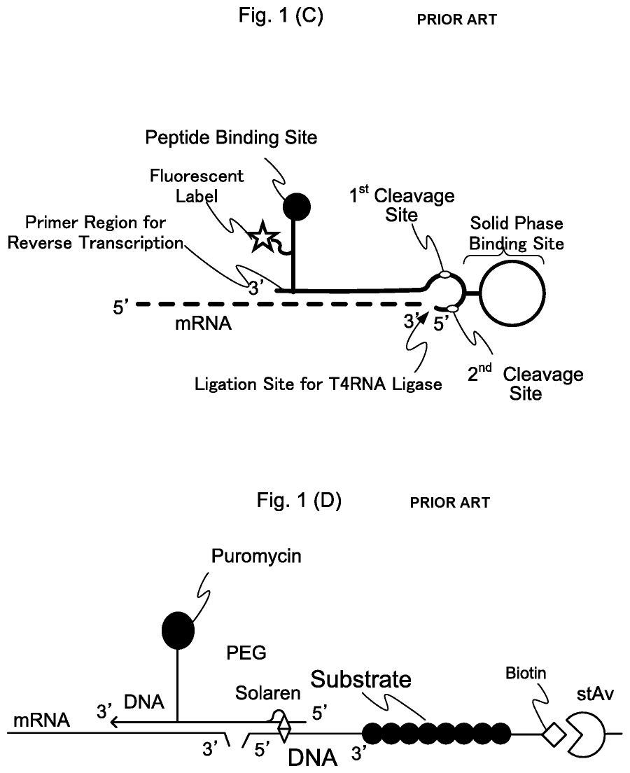

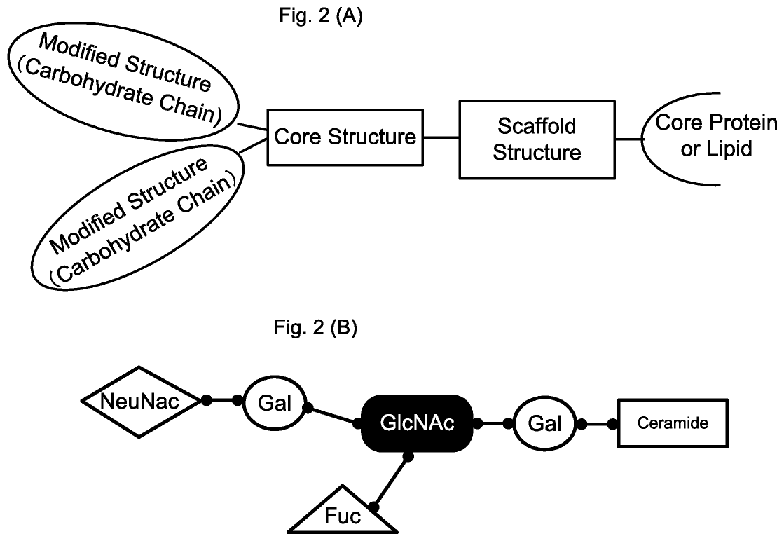

The structures of the linkers of the prior arts 1 to 5 (herein below, for example, it is referred to as “the prior linker 1”) are shown in FIGS. 1A to 1D.

### (Comparative Example 1) Synthesis of the Prior Linker 1 (SBP Linker) and Property Evaluation

### (1) Synthesis of the Prior Linker 1

Short-biotin-puromycin linker (SBP linker) was synthesized. Firstly, synthesis of the following (A) and (B1) were ordered to Gene world Inc. (Tokyo) and BEX Inc. (Tokyo).

### (A) Synthesis of Puro-F-S Segment [Sequence: 5′-(S)-(PL)C(F)-(Spacer18)-(Spacer18)-(Spacer18)-(Spacer18)-CC-(Puro)-3′]

Here, (S) is Thiol-Modifier C6 S-S(its compound name: o-(dimethoxytrityloxy-hexyl-dithiohexyl)-o′-(2-cyanoethyl)-N,N-diisopropyl-phosphoramidite); (PL) is PC Linker Phosphoramidite (its compound name: 3-(4,4′-Dimethoxytrityl)-1-(2-nitrophenyl)-1-propanyl-[(2-cyanoethyl)-(N,N-diisopropyl)]-phosphoramidite). (F) is Fluorescein-dT (its compound name: (5′-Dimethoxytrityloxy-5-[N-((3′,6′-dipivaloylfluoresceinyl)-aminohexyl)-3-acryimido]-2′-deoxyUridine-3′-succinoyl-long chain alkylamino). (Puro) means puromycin.

(Spacer 18) means Spacer Phosphoramidite 18 (its compound name: 18-O-Dimethoxytrityl-hexaethyleneglycol,1-[(2-cyanoethyl)-(N,N-diisopropyl)]-phosphoramidite) (all of them are provided by Glen Research).

(B1)Hybri segment [(26 mer) SEQ ID NO:. 12:
5′-CCGCBCRCCC CGCCGCCCCC CGDCC T-3′]

Here, D is an amino-modifier C6 dT (5′-Dimethoxytrityl-5-[N-(trifluoro acetyl-aminohexyl)-3-acrylimido]-2′-deoxyUridine,3′-[(2-cyanoethyl)-(N,N-diisopropyl)]-phosphoramidite); B is Biotin-dT (5′-Dimethoxytrityloxy-5-[N-((4-t-butylbenzoyl)-biotinyl)-aminohexyl)-3-acrylimido]-2′-deoxyUridine-3′-[(2-cyanoethyl)-(N,N-diisopropyl)]-phosphoramidite), and R is riboG (all of them are provided by Glen Research).

According to the following method, SBP linker was cross-linked (A) puro-F-S segment with (B2) Hybri segment synthesized in the example 1(1) to be purified and obtained. 10 nmol Puro-F-S was dissolved in 100 μL of 50 mM phosphate buffer (pH 7.0), to which 1 μL of 100 mM Tris[2-carboxy-ethyl] phosphine (TCEP, Pierce) was added (final conc. 1 mM); then it was stood at room temperature for 6 hours to reduce a tritiated mercapto group of Puro-F-S to thiol group. Immediately before the cross-linking reaction, TCEP was removed from the solution and desalted by using NAP-5 column equilibrated with 50 mM phosphate buffer (pH 7.0).

Both of 20 μL of 500 pmol/μL of Hybri and 20 μL of 100 mM cross linking agent (EMCS) were added to 100 μL of 0.2 M phosphate buffer (pH 7.0), and stirred well at 37° C. for 30 minutes. Then, the incubated solution was subjected to ethanol precipitation, and the reaction products were precipitated at 4° C. to remove un-reacted EMCS. The precipitate was washed with 500 μL of cold 70% ethanol, and dried under reduced pressure. Then, the dried precipitate was dissolved in 10 μL of 0.2 M phosphate buffer (pH 7.0).

The reduced Puro-F-S(until 10 nmol) was immediately added to the reaction products, stood at 4° C. for overnight. TCEP was added to the sample so as to become 4 mM at final concentration, and stood at 37° C. for 15 minutes to stop the cross-linking reaction through thiol substrate. Then, the mixture was subjected to ethanol precipitation at room temperature to have the precipitated the synthesized linker, and then, the unreacted Puro-F-S was removed. In order to remove the unreacted Hybri segment, and the EMCS cross-linking products thereof, they were purified by using HPLC under the following condition.

### (HPLC Conditions)

Column: COSMOSIL(registered trademark) 10×250 mm C18-AR-300 (Nakarai Teague)
Buffer A: 0.1 M TEAA(triethylammonium acetate)
Buffer B: 80% acetonitrile (diluted with ultra-pure water)
Flow rare: 0.5 ml/minute
Concentration gradient: the concentration of A and B in the mixed buffer was changed from 85:15 to 65:15 (ratios of A:B) (during 33 minutes).

The products fractionated by using HPLC was detected on 18% acrylamide gel (8 M urea, 62° C.) by using gel electrophoresis, and the target fractions were dried under the reduced pressure. After that, the dried fraction was dissolved with DEPC (Diethylpyrocarbonate) treated water, and prepared 10 pmol/μL solution. The obtained linker (hereinbelow, it is referred to as the “prior linker 1”.) is schematically shown in FIG. 1A.

(2) mRNA Synthesis

As the model mRNA, BDA (B domain of Protein A) was used. Both of T7 promoter sequence and translation acceleration sequence were added to the upstream of 5′ side of BDA gene (BDA gene; 192 nucleotide length); the spacer region, Histidine tag (His tag) and DNA to which a complementary sequence with puromycin linker was added (BDA whole; SEQ ID NO: 6:367 nucleotide lengths in sequence listing) was added to 3′ side down stream was synthesized by using PCR. After the purification, 5 to 30 pmol/μL of mRNA (BDA mRNA) was synthesized by using T7 RiboMAX Express Large Scale RNA Production System (Promega)) according to the attached protocol thereof.

### (3) The Property Evaluation of the Prior Linker 1

(3-1) Formation of the Conjugate Thereof with mRNA

The ligation reaction of T4 RNA ligase was conducted as follows. 1 to 6 volumes of mRNA, when that of the prior linker 2 equals to 1, was added, and the reaction was conducted to ligate in 20 μL of T4 RNA ligase buffer (10 mM MgCl₂, 10 mM DTT and 1 mM ATP containing 50 mM Tris-HCl (pH 7.5)). Annealing was conducted before adding enzymes, it was warmed at 90° C. for 5 minutes, and then, warmed at 70° C. for 5 minutes. Finally, it was stood at room temperature for 10 minutes. Then, it was placed on ice. Here, both of 1 μL of T4 polynucleotide kinase (10 U/μL) and 1 μL of T4 RNA ligase (40 U/μL) (both of them were from Takarabio) were added to the solution, and held at 25° C. for 15 minutes.

Production solution was divided into two portions. In order to compare them in following experiment steps, the sample, wherein 10 pmol of mRNA was used to the 10 pmol of the linker, was reacted in 40 μL size system which contains 2 fold amounts of all of the components (the reaction system R1).

### (3-2) Results of the Ligation Reaction

The ligation efficiency between mRNA and SBP linker were compared in the following mixture ratio, which was conducted by gel electrophoresis using 8 M urea denaturating 5% acrylamide gel at 65° C., under the conditions of 200 V for 25 minutes. The samples were dispensed 1 μL each, and it was mixed with 3 μL of the loading buffer and 2 μL of DEPC water, and then loaded on the gel. The results were shown in FIG. 20. The bands were detected by using the fluorescence of FITC bounds to the linker to detect the unreacted linker or the conjugate A.

The prior linker was added to each reaction system for ligation reaction, and the reaction mixtures were subjected to gel-electrophoresis as follows: 10 pmol of the prior linker 2 being added into 10 pmol of mRNA, which was reacted by using the 40 μL of the system (the lane 1, the reaction system R1); 6.6 pmol of that was treated as the same as described above (the lane 2, the reaction system R2); 3.3 pmol of that was treated as the same as described above (the lane 3, the reaction system 3); 1.66 pmol of that was treated as the same as described above (the lane 4, the reaction system R4); each amount of the prior linker 2 (FIG. 20). In FIG. 20, the arrow shows the unreacted linker, * shows the reaction products to which the linker was bound. From the results, the amounts of the synthesized conjugate A in each lane has no differences except the case of the mix ratio 1:1. It was observed that the ligation reaction was occurred in the same amount in each system.

### (Comparative Example 2) Synthesis of the Prior Linker 2 (LBP Linker) and Property Evaluation of the LBP Linker)

Long-Biotin-puromycin-linker (LBP linker) was synthesized as follows. Firstly, the synthesis of the (1-1) (A) and (B2) in Comparative Example were ordered to Geneworld Inc. and BEX Co. Ltd.

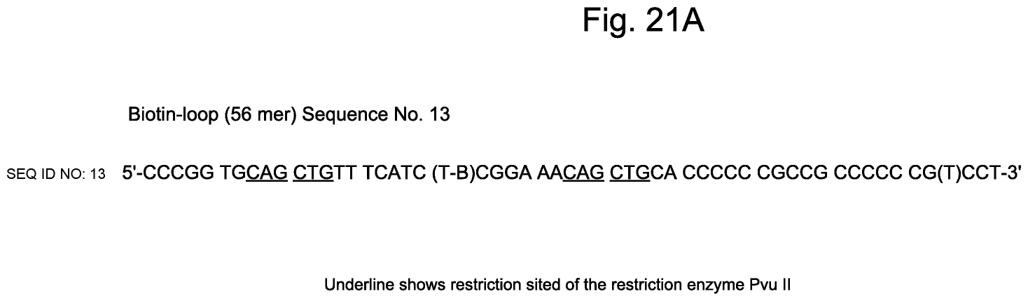

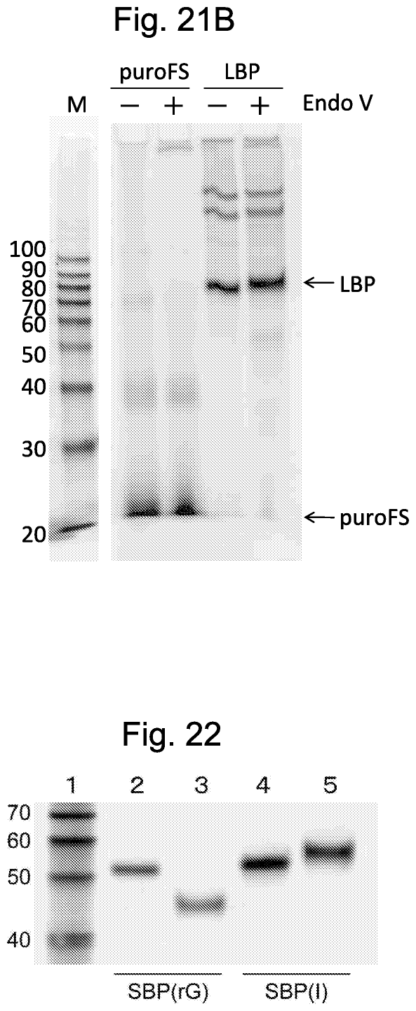

Restriction sites in the biotin-loop segment (56 mer) (SEQ ID NO: 13 in Sequence listing) were shown in FIG. 21A. Also, the chemical formula of thiol modifier C6 S-S was shown in the following formula (IX).

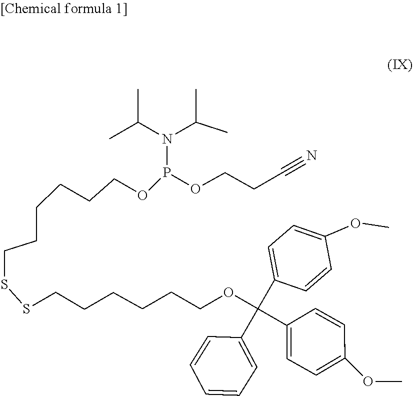 [SMILES: COc1ccc(C(OCCCCCCSSCCCCCCOP(OCCC#N)N(C(C)C)C(C)C)(c2ccccc2)c2ccc(OC)cc2)cc1]

The LBP linker was obtained by crosslinking of (A) Puro-F-S segment (the side chain) with (B) Biotin-loop segment (the molecular backbone) according to the following method, and then purified. Firstly, 10 nmol of Puro-F-S was dissolved in 22.5 μL of 1M phosphate buffer (pH 9.0), and 2.5 μL of 1M DTT was added, and then, the mixture was stood at room temperature for 1 hour to reduce trimehylated mercapto substitute in Puro-F-S to thiol substrate thereof. Immediately before the crosslinking reaction, the mixture was treated to remove DTT and to be desalted by using NAP-5 Columns (GE Healthcare) equilibrated with 20 mM phosphate buffer (pH 7.2).

Both of 10 μL of 500 pmol/μL Biotin-loop and 10 μL of 100 mM EMCS (6-maleimidehexanoic acid N-hydroxysuccinimide ester: the crosslinking agent, Dojindo Molecular Technology Inc.) were added into 50 μL of 0.2M phosphate buffer (pH 7.2), and stirred well, then the mixture was reacted at 37° C. for 30 minutes. After that, the products in the mixture were subjected to ethanol precipitation at 4° C. to remove the unreacted EMCS. The precipitate was washed with 500 μL of 70% ethanol, and then it was dried under reduced pressure.

The obtained products were quickly dissolved in the solution containing the reduced Puro-F-S(to 10 nmol), and then it was stood at 4° C. for overnight to prepare the sample. DTT was added to the sample so as that final concentration thereof became 50 mM, and stood at 37° C. for 30 minutes to terminate the crosslinking reaction by the thiol substitute. By using the ethanol precipitation method, the synthesized linker was precipitated at room temperature to remove the unreacted Puro-F-S. Further, in order to delete the unreacted Biotin-loop and the crosslinked substances thereof and EMCS, the products were purified by using the gradient method of HPLC under the following conditions.

### (HPLC) Conditions

Column: Symmetry 300 C18, 5 m, i.d. 4.6 mm×250 mm (Waters Corporation)
Elution buffer: Solution A and Solution B were used as mixed solution in below.
   Solution A: 0.1 M TEAA (triethylamnonium acetate)
   Solution B: 80% acetonitrile (diluted with ultra-pure water)
Flow rate: 0.5 mL/minute
Gradient of the elution buffer: The ratio of Solution A: Solution B was changed from 85:15 to 65:35 during 30 minutes.

The fractionated products by using HPLC were detected in gel electrophoresis by using 16% acrylamide gel (8 M urea, 60° C.), and the fractions of the interest were dried under the reduced pressure. After that, it was dissolved in DEPC (bicarbonate diethyl, diethylpyrocarbonate)treated water so as to become 10 pmol/μL.

### (2) Property Evaluation of the Prior Linker 2

Cleavage of the prior linker 2 by Endonuclease V (hereinbelow, it is sometimes referred to as “Endo V”. M0305S, New England Biolabs, Inc. (hereinbelow, it is referred to as “NEB”.)) was evaluated.

The reaction was conducted in 10 μL of the mixture containing 10 pmol of the prior linker 1, 0.5 μL of Endo V (10 U/μL), 1 μL of 10×NE buffer and distilled water at 37° C., for 30 minutes. After termination of the reaction, the mixture was desalted with P6 column (Bio-Rad Laboratories, Inc.). As the prior linker 1, the solution equivalent to 5 pmol was taken and analyzed by the method of SDS-PAGE, which was conducted for 200 V, 30 minutes under the conditions of 12% polyacrylamide gel. After finishing the gel electrophoresis, the gel was stained by using SYBR(registered trademark) Gold, and then the electrophoresis profile was observed by using fluorescence. As a result, it was confirmed that no products cleaved by Endo V were detected (see FIG. 21B).

### (Comparative Example 3) Synthesis of the Prior Linker 3 and the Property Evaluation Thereof

### (1) Synthesis of the Prior Linker 3

Both of inosine-Short-Biotin-puromycin linker (SBP (I) linker) and rG-Short-Biotin-puromycin⋅linker (SBP (rG) linker) used in the experiment were synthesized as follows. Firstly, in addition to (A) and (B1) described in the comparative example 1, the synthesis of two particular DNAs, (C1) I-hybri segment ((28 mer), SEQ ID NO: 14 in sequence listing) and (C2) rG-Hybri segment ((26 mer) SEQ ID NO: 15 in sequence listing), were ordered to Geneworld (Tokyo).

Here, in (B1), I represents deoxy inosine, symbols represent (T), (T-B) and modified nucleotide are the same as those used in the comparative example 1 and 2. Also, in (C1), (rG) represents riboG.

The SBP (I) linker was obtained by using (A) Puro-F-S segment and (B) I-Hybri segment, both of which were synthesized in Example 1 (1), and were crosslinked according to the following method and then purified. Also, the SBP (rG) linker was obtained by crosslinking of (A) Puro-F-S and (C) rG-hybri.

1.7 μL of 3 mM Puro-F-S was mixed with 22.5 μL of 1M phosphate buffer (pH 9.0), and 2.5 μL of 1M DTT was added, and stood at room temperature for 1 hour to reduce the tritylated mercapto substitute to thiol substitute. Immediately before conducting the crosslinking reaction, DTT was removed from the mixture by using NAP-5 Columns (GE Healthcare) equilibrated with 20 mM (pH 7.2) and desalting.

2.5 μL of 1 mM I-Hybri or rG-Hybri, and 10 μL of 100 mM EMCS were added to 50 μL of 0.2 M phosphate buffer (pH 7.2) and stirred well to react them at 37° C. for 30 minutes. After that, the reaction products were subjected to ethanol precipitation to remove the unreacted EMCS. Then, the obtained precipitate was washed with 200 μL of 70% alcohol and dried under the reduced pressure.

The reaction products was quickly dissolved in the reduced Puro-F-S solution (to 5 nmol), and stood at 4° C. for overnight to prepare the sample. DTT was added to the sample so as that its final concentration become 50 mM, and stood at 37° C. for 30 minutes to terminate the crosslinking reaction of the thiol substrate. Then, the synthesized linker was precipitated by using ethanol precipitation at room temperature to remove the unreacted Puro-F-S. After that, in order to further remove the unreacted I-Hybri or unreacted rG-Hybri, and EMCS cross-liked products thereof, the sample was subjected to the purification by using HPLC under the same conditions as those in Example 1.

The fractionated products by HPLC was subjected to gel electrophoresis by using 12% acrylamide gel containing 8 M urea under the conditions of 200 V, at 60° C. for 30 minutes for fractionation. The fractions of the interest were dries under the reduced pressure. After that, it was dissolved in DEPC (Diethylpyrocarbonate) treated water to dilute 10 pmol/μL.

### (2) RNase Resistant Test of the Linker

RNase resistant test of both linkers, SBP (rG) and SBP (I) synthesized as described above was conducted by using RNase ONE (Promega) derived from Escherichia coli (E. coli) periplasm. RNase ONE is the RNA degrading enzyme, which has the activity to cleave phosphodiester bond positioned at 3′ terminal side of each RNA, namely A, C, G, and U.

Either 1 pmol of SBP (rG) or SBP(I), 0.5 μL of RNase ONE (10 U/μL), 1 μL of 10× RNase ONE reaction buffer (Promega), and RNase free water were added to mix to prepare 10 μL of the mixture.

Then, the mixture was reacted at 37° C. for 30 minutes, and the reaction products were analyzed by using SDS-PAGE method. Gel electrophoresis was conducted by using 12% acrylamide gel containing 8 M urea under the conditions of 200 V, at 60° C. for 30 minutes. Then, FITC contained in the linker was detected by laser excitation fluorescence of which excitation wave length was 488 nm with Molecular Imager Pharos FX (Bio-Rad Laboratories, Inc.). The results were shown n FIG. 21B.

In FIG. 21B, 1 μL of 100 bp DNA ladder (Promega) was applied as a size marker at the 1^{st} lane. 0.5 pmol of the unreacted SBP (rG) was applied to the 2^{nd} lane, and 5 L of SBP (rG) (in the figure, it was shown as “puroFS”) treated with RNase ONE was applied to the 3^{rd} lane, 0.5 pmol of untreated SBP (I) was applied to the 4^{th} lane, and 5 L of RNase ONE treated-SBP (I) was applied to the 5^{th} lane.

In FIG. 22, compare to the 2^{nd} lane, to which the untreated SBP (rG) was applied, with the 3^{rd} lane, to which RNase ONE treated-SBP (rG) was applied, the band positions in the 3^{rd} lane were shifted to low molecular weight direction. Therefore, this showed that SBP (rG) was cleaved at the riboG site by RNase ONE. On the other hand, compared to the 4^{th} lane, to which the untreated SBP (I) was applied, with the 5^{th} lane, to which RNase ONE-treated SBP (I) was applied, no band positions shit to the lower molecular weight direction were observed. Therefore, this showed that SBP (I) was not cleaved by RNase ONE.

According to the tests described above, it was showed that SBP (I) has ribonuclease resistance which was lacked in the prior SBP (rG) linker.

### (Comparative Example 4) Synthesis of the Prior Linker 4 and the Property Evaluation

### (1) Synthesis of the Prior Linker 4

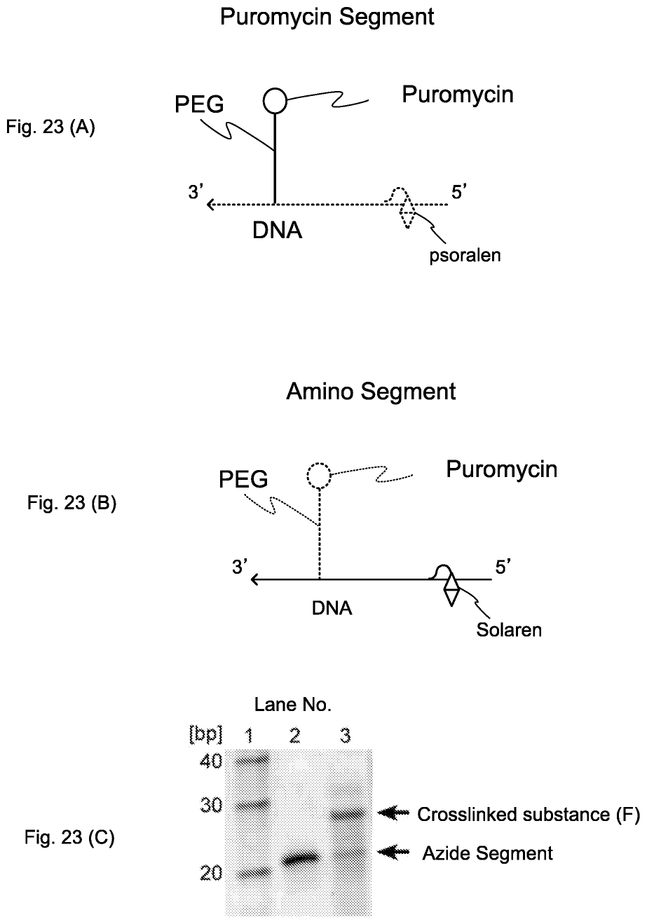

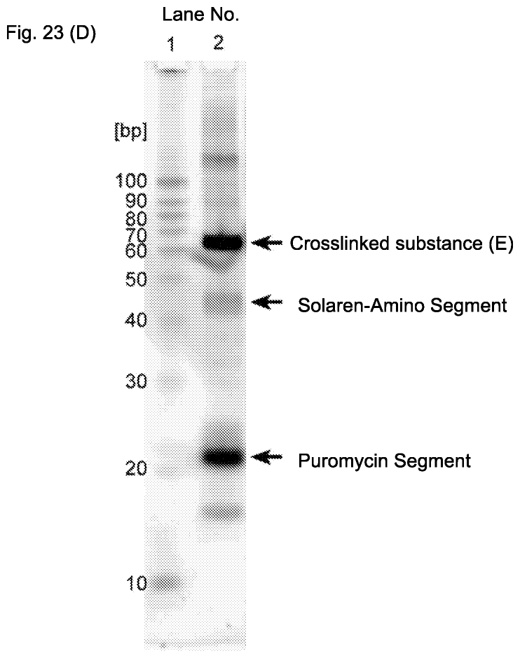

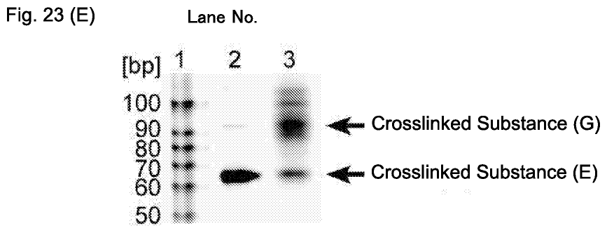

The prior linker 4 (FIG. 1D) was synthesized by using following 4 components: (a) puromycin segment (FIG. 23A), (b) Solaren-amino segment (FIG. 23B), (c) azide segment, and (d) alkyne-peptide-biotin segment. Among them, (a) and (b) were used for the synthesis of the molecular backbone, and (c) and (d) were used for that of the substrate unit. Special DNAs employed for the synthesis of (a) to (c) segments were ordered to Geneworld and Japan Bioservice. The synthesis of peptides for (d) was ordered to Scrum Inc. As the (a) puromycin segment, the peptides synthesized in Example 2 (2-1) were used.

### (2) Synthesis of Solaren-Amino Segment

Solaren-amino segment having the following configuration (SEQ ID NO: 16 in sequence in sequence listing) was synthesized. Here, as the (psolaren), Psolaren C6 Phosphoramidite ((6-[4′-(Hydroxymethyl)-4,5′,8-trimethylpsoralen]-hexyl-1-O-(2-cyanoethyl)-(N,N-diisopropyl)-phosphoramidite)) was used.

[SEQ ID NO: 16]
5′-(psoralen)-TACGACGATCTCGAACGAACCA
CCCCCGCCGCCCCCCG-(T-NH2)-CCT-3′

Also, as (T-NH2), Amino-Modifier C6 dT(5′-Dimethoxytrityl-5-[N-(trifluoro acetylaminohexyl)-3-acrylimido]-2′-deoxyUridine, 3′-[(2-cyanoethyl)-(N,N-diisopropyl)]-phosphoramidite) was used. Those reagents were purchased from Glen Research. Those regents were reacted according to phosphoramidite method with an automated polynucleotide synthesizer (FIG. 23B).

### (3) Synthesis of Azide Segment

The azide segment having the following structure (SEQ ID NO: 17 in sequence listing) was synthesized. Here, as (Spacer 18), Spacer Phosphoramidite 18((18-O-Dimethoxytrityl hexaethyleneglycol, 1-[(2-cyanoethyl)-(N,N-diisopropyl)]-phosphoramidite)) was used.

SEQ ID NO: 17]
5′-CCCGTGGTTCGTTCGAGATCGTCGTAAA-3′

AAA at the 3′-terminal of the sequence was bound to -(Spacer 18)-(C6)-(Azide); wherein as (C6), 3′-Amino-Modifier C7 CPG 500(2-Dimethoxytrityloxymethyl-6-fluorenyl methoxycarbonylamino-hexane-1-succinoyl-long chain alkylamino-CPG) was used.

As (Azide), Azide butyrate NHS ester (4-Azido-butan-1-oic acid N-hydroxy succinimide ester) was used. Those reagents were purchased from Glen Research. Those reagents were reacted to synthesize the azide segment according to phosphoramidite method with an automated polynucleotide synthesizer.

### (4) Synthesis of Alkyne-Peptide-Biotin Segment

Alkyne-peptide-biotin segment having the following configuration (SEQ ID NO: 18 in sequence listing) was synthesized. The following sequences were shown in the structure from N-terminal to C terminal. Here, as Gly (Propargyl), Fmoc-Gly(Propargyl)-OH was used. In (K-biotin)-NH₂, C terminal of lysine residue was amidited, and the side chain of the lysine residue was modified by binding to biotin.

### [SEQ ID NO: 18]

Gly(Propargyl)-EDHVAHALAQ-(K-Biotin)-NH₂
Azide shown in the following formula (I)

[Chemical formula 2]

5′-DNA-3′-(EG)₅-C₇—N≡N (II)

and alkyne shown in the following formula (II)

[Chemical formula 3]

C≡C [symbol] N [symbol] Peptide [symbol] C)-Biotin (II)

were mixed at the molar ratio of 1:100, and reacted for 64 hours by using CuSO₄/Ascorbate as a catalyst to obtain the reaction product shown in the following formula (III).

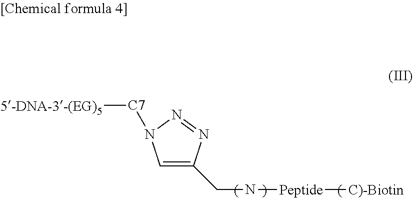

### (5) Crosslinking of the Azide Segment and the Alkyne-Peptide-Biotin Segment

L of 25 μM azide segment and 25 μL of 1 mM alkyne-peptide-biotin segment were mixed with the solution composed of 60 μL of t-butanol, 5 μL of 0.5 mM CuSO₄, and 5 μL of 2.5 mM ascorbic acid ester, and conducted crosslinked reaction with stirring at room temperature for 64 hours. The mixture containing the resulting cross-linked products was desalted with the micro bio spin column 6 (Bio-Rad) equilibrated by using phosphate buffer (pH 7.2). Then, the desalted cross-linked products were separated by using polyacrylamide gel electrophoresis, and separated DNAs were stained by using SybrGold (Invitrogen) to analyze.

The results were shown in FIG. 23C. The lane 1 shows 10 bp of DNA step ladder (Promega), the lane 2 shows the azide-segment, and the lane 3 shows cross-linked products. From these results, it was confirmed that the cross-linked products of the interest (F) was obtained about 70% yield (FIG. 23C).

### (6) Crosslinking of the Puromycin Segment and Solaren-Amino Segment

5 μL of 4 mM puromycin segment was mixed with 45 μL of 1 M phosphate buffer (pH 9.0), and then 5 μL of 1M DTT was added, and stood at room temperature for 1 hour to reduce the disulfide group in the puromycin segment to thiol group. Immediately before the cross-linking reaction, DTT and salts were removed from the mixture by using NAP-5 Column equilibrated with 20 mM phosphate buffer (pH 7.2). 10 μL of 1 mM solaren-amino segment and 20 μL of 100 mM the cross linking agent (EMCS) were added to 100 μL of 0.2M phosphate buffer (pH 7.2) and stirred well for reacting at 37° C. for 30 minutes.

Then, the reaction products were precipitated according the conventional ethanol precipitation method to remove the unreacted EMCS. The precipitate was washed with 200 μL of 70% ethanol, and dried under the reduced pressure. The reaction products were quickly dissolved in the puromycin segment solution which was reduced as described above (about 20 nmol), and stood at 4° C. for overnight. After that, DTT was added so as that the final concertation thereof become 50 mM, and stood at 37° C. for 30 minutes to terminate the cross-linking reaction of thiol group. Then, the reaction mixture including the cross-liked products wherein two segments were cross-linked.

The obtained cross-linked products were separated by using urea denaturing polyacrylamide gel-electrophoresis (12% acrylamide gel, 8 M urea, 60° C.), and then separated DNAs were stained with SybrGold (Invitrogen) to analyze. The lane 1 shows the 10 bp of DNA step ladder (Promega), the lane 2 shows the results of the cross-linked products. Except bands from puromycin segment and solaren-amino segment, the bands which have the molecular weight comparative to the total of these two segments. Therefore, it was confirmed that the cross-linked products of the interest (E) was obtained. The results were show in the FIG. 23D.

Next, the cross-linked products (E) were precipitated by using ethanol to remove the unreacted puromycin segment. In order to further remove the unreacted solaren-amino segment and EMCS cross-linked products thereof, the purification was conducted under the following conditions by using HPLC.

### (HPLC) Conditions

Column: Symmetry 300 C18, 5 m, i.d. 4.6 mm×250 mm (Waters Corporation)
Elution buffer: Solution A and Solution B were used as mixed solution in below.
   Solution A: 0.1 M TEAA (triethylammonium acetate)
   Solution B: 80% acetonitrile (diluted with ultra-pure water)
Flow rate: 0.5 mL/minute
Gradient of the elution buffer: The ratio of Solution A:Solution B was changed from 85:15 to 50:50 during 50 minutes.

The products fractionated by using HPLC was again detected by using gel electrophoresis with 12% acrylamide gel (8 M urea, 60° C.), and the fractions of the interest were condensed under the reduced pressure. Then, it was subjected to ethanol precipitation as the same conditions described above. After that, the precipitate was dissolved with water and diluted to 50 μM.

### (7) The Photo-Cross-Linking of the Crosslinked Products (E) and (F)

1 pmol of the cross-linked products (E) and 1.2 pmol of those (F) were mixed in the solution containing 20 mM Tris-HCl (pH 8.0) and 100 mM NaCl. Then, the solution was heated to 60° C. and cooled to 25° C. during 10 minutes. Next, the light having the wave length of 365 nm (2 W/cm²) of which light source was Xenon lamp was irradiated for 20 minutes for exposing the sample to prepare the light exposed sample. The light exposed sample was separated by using urea denaturing polyacrylamide gel electrophoresis under the same conditions as described above. Then DNAs in the gel was stained by using SybrGold (Invitrogen) to analyze. The results were shown in FIG. 23E.

As shown in FIG. 23E, the light exposed sample showed the band of the electrophoresis which was shifted to long chain direction, and it was confirmed that the crosslinked products (G) were generated. HPLC analysis results also confirmed that the crosslinked products were eluted at the retention time of 31.819 minute.

### INDUSTRIAL APPLICABILITY

The present invention is available in the technical fields of pharmaceutical preparation by using a molecular targeting type peptide pharmaceutical preparation, a low molecular weight antibody and antibody like protein
SEQ ID NO: 1: Nucleotide sequence of cnvK linker backbone sequence
SEQ ID NO: 2: Nucleotide sequence of a primer (T7Ωnew)
SEQ ID NO: 3: Nucleotide sequence of the primer (NewYtag)
SEQ ID NO: 4: Nucleotide sequence of the primer (Newleft)
SEQ ID NO: 5: Nucleotide sequence of B domain of A protein
SEQ ID NO: 6: VHH library construct
SEQ ID NO: 7: Random library sequence employed in in vivo selection method
SEQ ID NO: 8: ΩRT-L new
SEQ ID NO: 9: NewYtag (22 mer)
SEQ ID NO: 10: DNA sequence of PDO
SEQ ID NO: 11: Nucleotide sequence of FLAG
SEQ ID NO: 12: Nucleotide sequence of Hybri segment in prior linker
SEQ ID NO: 13: Nucleotide sequence of the backbone in the prior linker
SEQ ID NO: 14: I-hybri segment in the prior linker
SEQ ID NO:15: Nucleotide sequence of rG-hybri segment in the prior linker
SEQ ID NO: 16: Nucleotide sequence of solaren-amino segment in the prior linker
SEQ ID NO: 17: Nucleotide sequence of azide segment in the prior linker
SEQ ID NO: 18: Nucleotide sequence of biotin segment of the side chain in the prior linker

Sequence Listing

1. A method for in vitro selection comprising the steps of:
forming a complementary bond for binding a molecular backbone of a high-speed photo-cross-linking shared linker for in vitro selection and intermolecular interaction analysis to a desirable mRNA;
photo-cross-linking by using irradiation of light having 300 to 500 nm wavelength for 0.01 to 5 minutes to both of said molecular backbone and mRNA which are mutually bound through a complementary bond;
forming a fusion body being composed of mRNA-protein, wherein the protein is obtained through translation of mRNA bound to the linker in cell-free translation system and said protein is bound to the linker;
binding said fusion body to a solid phase;
reverse-transcribing a mRNA included in the fusion body to obtain cDNA and to form a conjugate being composed of the fusion body and reverse-transcribed cDNA; and
choosing desirable cDNA through cleaving the fusion body from the solid phase,
wherein the high-speed photo-cross-linking shared linker for in vitro selection and intermolecular interaction analysis comprises the molecular backbone and a side chain:
said molecular backbone comprising,
a solid phase binding site having a predetermined nucleotide sequence and located at 5′ end thereof for forming a bond to bind to said solid phase;
a solid phase cleavage site for cleaving said solid phase including said solid phase binding site;
a side chain ligation site for ligating said side chain to said molecular backbone;
a high-speed photo-cross-linking site locating between said side chain binding site for ligating mRNA having a complementary sequence with that of the molecular backbone by using photo-cross-linking to said molecular backbone; and
a reverse transcription starting region adjacent to said side chain binding site and locating at 3′ end of the molecular backbone;
said side chain comprising a fluorescent label, a protein fusing site locating at a free end thereof, and a ligation formation site for being bound to said molecular backbone; and
said side chain is ligated to said side chain ligation site at the ligation formation site in the molecular backbone.

2. The method for in vitro selection according to the claim 1, wherein said solid phase is composed of a magnetic bead coated by either streptavidin or avidin.

3. The method for in vitro selection according to the claim 1, wherein said cleavage of the conjugate is conducted by using any one of the enzyme selected from the group consisting of endonuclease V, Rnase T1, and RNase A.

4. The method for in vitro selection according to claim 1, wherein the molecular backbone of the high-speed crosslinking shared linker comprises a sequence for recognizing a carbohydrate antigen.

5. A method for preparing a linker-protein for affinity measurement comprising the steps of:
forming a complementary bond for binding a molecular backbone of a high-speed photo-cross-linking shared linker for the in vitro selection and intermolecular interaction analysis to a desirable mRNA;
photo-cross-linking by using irradiation of light having 300 to 400 nm wavelength for 0.05 to 5 minutes to both of said molecular backbone and mRNA which are mutually bound through a complementary bond;
forming a fusion body being composed of mRNA-protein, wherein the protein is obtained through translation of mRNA bounds to the linker in cell-free translation system and said protein is bound to the linker;
forming a fusion body being composed of the linker-protein by treatment of RNA digestion of the fusion body being composed of mRNA-protein;
binding said fusion body being composed of linker-protein to a solid phase; and
purifying said fusion body being composed of linker-protein eluted from said solid phase under a predetermined condition,
wherein the high-speed photo-cross-linking shared linker for in vitro selection and intermolecular interaction analysis comprises the molecular backbone and a side chain:
said molecular backbone comprising,
a solid phase binding site having a predetermined nucleotide sequence and located at 5′ end thereof for forming a bond to bind to said solid phase;
a solid phase cleavage site for cleaving said solid phase including said solid phase binding site;
a side chain ligation site for ligating said side chain to said molecular backbone;
a high-speed photo-cross-linking site locating between said side chain binding site for ligating mRNA having a complementary sequence with that of the molecular backbone by using photo-cross-linking to said molecular backbone; and
a reverse transcription starting region adjacent to said side chain binding site and locating at 3′ end of the molecular backbone;
said side chain comprising a fluorescent label, a protein fusing site locating at a free end thereof, and a ligation formation site for being bound to said molecular backbone; and
said side chain is ligated to said side chain ligation site at the ligation formation site in the molecular backbone.

6. The method for preparing a linker-protein for affinity measurement according to the claim 5, wherein said solid phase is composed of a magnetic bead coated by either streptavidin or avidin.

7. The method for preparing a linker-protein for affinity measurement according to the claim 5, wherein said purification step is conducted in an aqueous solution including 1 to 100 mM NaCl at room temperature.

8. A linker-protein for affinity measurement prepared by using any one of the method according to the claim 5.

9. The method for in vitro selection according to the claim 2, wherein said cleavage of the conjugate is conducted by using any one of the enzyme selected from the group consisting of endonuclease V, Rnase T1, and RNase A.

10. The method for in vitro selection according to claim 2, wherein the molecular backbone of the high-speed crosslinking shared linker comprises a sequence for recognizing a carbohydrate antigen.

11. The method for preparing a linker-protein for affinity measurement according to the claim 6, wherein said purification step is conducted in an aqueous solution including 1 to 100 mM NaCl at room temperature.
